(19) United States
(12) Patent Application Publication
(10) Pub. No.: US 2026/0069635 A1
(43) Pub. Date: Mar. 12, 2026

(54) MESENCHYMAL PROGENITOR CELLS FOR ENHANCING PARTIAL REPROGRAMMING OF TARGET CELLS

(71) Applicant: Mesoblast International Sarl, Meyrin (CH)

(72) Inventor: Silviu ITESCU, Melbourne (AU)

(21) Appl. No.: 18/872,445

(22) PCT Filed: Jun. 8, 2023
(86) PCT No.: PCT/IB2023/055908

§ 371 Date: Dec. 6, 2024

(30) Foreign Application Priority Data
Jun. 8, 2022 (AU) 2022901577

(51) Int. Cl. A61K 35/28; A61K 31/5575; A61K 31/7105; A61K 38/00; A61K 48/00; C07K 16/28; C12N 5/0775; C12N 5/10; C12N 15/861

(52) U.S. Cl. CPC A61K 35/28; A61K 31/5575; A61K 31/7105; A61K 38/00; A61K 48/005; C07K 16/28; C12N 5/0663; C12N 5/10; C12N 15/8616; C12N 2501/24; C12N 2501/25; C12N 2506/1353; C12N 2510/00; C12N 2710/10043

(57) ABSTRACT

Provided herein are methods and related compositions for enhancing or enhanced partial reprogramming of target cells in a subject in need thereof (e.g., a human subject suffering from or at risk of a disease), the method comprising administering a plurality of mesenchymal lineage progenitor or stem cells (MLPSCs), exosomes derived therefrom, or conditioned culture media derived therefrom to a subject that expresses or will express one or more reprogramming factors in a population of target cells, whereby a plurality of the target cells in the subject become partially reprogrammed, but not fully reprogrammed.

The present application claims priority from AU 2022901577 filed on 8 Jun. 2022, the entire content of which is incorporated herein by reference.

### FIELD OF THE INVENTION

The present invention relates to methods and related compositions for enhancing partial reprogramming and therapeutic applications thereof.

### BACKGROUND OF THE INVENTION

A wide range of differentiated mammalian cell types can reacquire broad developmental potential when experimentally induced to express one or more “reprogramming factors.” When such reprogramming factors are expressed in differentiated cells for a sufficient period of time, the differentiated cells are reprogrammed into pluripotent stem cells termed “induced pluripotent stem cells” (iPSCs). Perhaps one of the most remarkable features of the reprogramming process is that, in addition to “resetting” cell fate, cell “age” (relative to cell donor age) is also reset, yielding rejuvenated cells, as reflected by a range of characteristics, e.g., telomere length, epigenetic changes, and gene expression changes. Indeed, human iPSC lines can be generated from individuals from young to elderly, and the determined age of hiPSCs from these lines is effectively equivalent to embryonic age.

It has been shown that during the process of cell reprogramming resetting of cell age occurs prior to loss of differentiated cell identity. Thus, by limiting reprogramming factor expression time, differentiated cells are rejuvenated without losing cell type identity, a process referred to as “partial reprogramming.” A broad range of health conditions are associated with damaged and/or senescent tissues (e.g., cardiovascular diseases, low back pain, and osteoarthritis). While partial reprogramming of cells in vivo in animal models has been achieved, the exceedingly low efficiency of the process is a serious barrier to the potential therapeutic of partial reprogramming. Thus, there is an ongoing need for methods and compositions for enhancing partial reprogramming.

### SUMMARY OF INVENTION

Mesenchymal lineage progenitor or stem cells (MLPSCs) such as human mesenchymal stem cells (hMSCs) and human mesenchymal progenitor cells (hMPCs) have a very broad range of therapeutic properties and uses. Among these are their remarkable anti-inflammatory attributes, e.g., their ability to downregulate pro-inflammatory cytokine levels, as well as modulating immune cell responses, e.g., down-regulation of mTOR activity in T cells and effector functions in natural killer (NK) cells. While not wishing to be bound by theory, it is believed that several of the cell signalling pathways and immune cell effector functions that MLPSCs downregulate are ones that inhibit and reduce the efficiency of cell reprogramming including partial reprogramming, and may be particularly important in the context of partial reprogramming in subjects of advanced age in which a pro-inflammatory milieu is found in a number of tissues. In addition, in some embodiments, MLPSCs can damp an immune response to expression vectors (e.g., recombinant viruses) useful for in vivo delivery of reprogramming factors or inducing their expression in a population of target cells. Further, some therapeutic effects provided by MLPSCs can also be provided by exosomes isolated from MLPSCs. Thus, it is believed that the efficiency of cell reprogramming will be enhanced in the presence of MLPSCs or exosomes derived from MLPSCs.

Accordingly, in one aspect provided herein is a method for enhancing partial reprogramming of target cells in a subject in need thereof, the method comprising administering a plurality of mesenchymal lineage progenitor or stem cells (MLPSCs), exosomes derived therefrom, or conditioned media derived from culture of a plurality of MLPSCs to a subject that expresses or will express, following the administration, one or more reprogramming factors in a population of target cells, whereby a plurality of the target cells in the subject become partially reprogrammed.

In some embodiments the population of target cells comprises one or more exogenous nucleic acids encoding the one or more reprogramming factors.

In other embodiments the population of target cells comprises one or more exogenous nucleic acids encoding one or more targeted transactivators for inducing endogenous expression of the one or more reprogramming factors.

In some embodiments of the methods described herein, the MLPSCs are a population of cells culture expanded from a population of human STRO-1⁺ or TNAP⁺ mesenchymal progenitor cells (MPCs), or from a population of human mesenchymal stem cells (hMSCs). In some embodiments the MLPSCs are pre-licensed MLPSCs. In some embodiments, where the MLPSCs are pre-licensed MLPSCs, the pre-licensed MLPSCs are MLPSCs that were culture expanded in media containing: IFN-gamma and/or TNF-alpha; and one or more pro-inflammatory cytokines selected from the group consisting of IL-6, IL-8, IL-17A, MCP-1-alpha, MIP-1-beta, and IP-10. In some embodiments the media contains serum that contains IFN-gamma and/or TNF-alpha; and the one or more of the above-mentioned pro-inflammatory cytokines. In some embodiments, where the culture media contains serum, the culture medium contains newborn mammalian serum. In some embodiments the newborn mammalian serum is newborn calf serum.

In some embodiments the MLPSCs are MLPSCs genetically modified to overexpress indoleamine 2,3-dioxygenase (IDO) and/or cyclooxygenase-2 (COX-2).

In some embodiments the method comprises administering the plurality of MLPSCs. In other embodiments the method comprises administering the exosomes derived from a plurality of MLPSCs. In other embodiments the method comprises administering the conditioned media derived from culture of a plurality of MLPSCs.

In some embodiments the subject to whom MLPSCs, exosomes thereof, or culture medium is to be administered is suffering a health condition to be treated selected from the group consisting of: cardiovascular diseases, vascular endothelial conditions, low back pain, inflammatory diseases, osteoarthritis, metabolic diseases, kidney diseases, liver diseases, and neurological disorders. In some embodiments the health condition is a cardiovascular disease, low back pain, or an inflammatory disease.

In some embodiments the target cells to be partially reprogrammed in the subject are selected from the group consisting of: cardiomyocytes, nucleus pulposus cells, renal cells, chondrocytes, liver cells, endothelial progenitors, chondrogenic progenitors, neurons, and glial cells.

In some embodiments of the methods disclosed herein the one or more reprogramming factors are selected from the group consisting of: OCT4, SOX2, KLF4, c-MYC, LIN28, and NANOG. In some embodiments the one or more reprogramming factors comprise OCT4, SOX2, and KLF4. In other embodiments the one or more reprogramming factors comprise OCT4, SOX2, KLF4, and c-MYC. In some embodiments the one or more reprogramming factors comprise OCT4, SOX2, and KLF4, but not c-MYC. In other embodiments the one or more reprogramming factors: (i) do not comprise LIN 28; or (ii) do not comprise NANOG. In some embodiments the one or more reprogramming factors comprise OCT4, SOX2, KLF4, c-MYC, LIN28, and NANOG. In other embodiments the one or more reprogramming factors consist of: (i) OCT4, SOX2, KLF4, c-MYC, LIN28, and NANOG; (ii) OCT4, SOX2, KLF4, and c-MYC; or (iii) OCT4, SOX2, and KLF4.

In some embodiments the one or more exogenous nucleic acids in the target cells comprise one or more synthetic mRNAs encoding the one or more reprogramming factors or encoding the one or more targeted transactivators. In some embodiments the one or more synthetic mRNAs comprise one or more nucleoside-modified mRNAs. In other embodiments the one or more synthetic mRNAs are nonmodified synthetic mRNAs.

In some embodiments the one or more exogenous nucleic acids comprise one or more non-integrated expression cassettes for expression of the one or more encoded reprogramming factors or targeted transactivators in mammalian cells. In some embodiments, where the one or more exogenous nucleic acids comprise one or more non-integrated expression cassettes, the one or more non-integrated expression cassettes are provided as one or more: a plasmid expression vectors, minicircle expression vectors, linear amplicons, circular DNA amplicons, a recombinant DNA viral genomes or amplicons, or recombinant RNA viral genome or amplicons. In some embodiments, where non-integrated expression cassettes are used, at least one of the one or more encoded reprogramming factors or targeted transactivators is under the control of a target cell-selective promoter. In some embodiments the target cell-selective promoter is selected from the group consisting of: cardiomyocyte-selective promoters, nucleus pulposus cell-selective promoters, renal cell-selective promoters, liver cell-selective promoters, endothelial progenitor-selective promoters, chondrogenic progenitor-selective promoters, glial cell-selective promoters, and neuron-selective promoters.

In some embodiments of any of the foregoing methods at least at least one of the exogenous nucleic acids is polycistronic.

In some embodiments expression of the one or more reprogramming factors is inducible and reversible. In some embodiments inducible and reversible expression is regulated by a reverse tetracycline transactivator (rtTA).

In some embodiments the MLPSCs to be administered are modified MLPSCs comprising at least one of: an exogenous anti-inflammatory: miRNA, antagomiR, siRNA, RNAi, antisense oligonucleotide, or an antisense RNA. In some embodiments, MLPSCs are modified MLPSCs comprising at least one of a: miRNA, antagomiR, siRNA, RNAi, antisense oligonucleotide, or antisense RNA directed against at least one of: p16^{INK4a}, the mTOR signalling pathway, or the c-Jun N-terminal kinase (JNK) signalling pathway.

In some embodiments the one or more reprogramming factors are expressed consecutively for at least two days, but not longer than about 15 days.

In some embodiments the plurality of MLPSCs are administered to the subject after expression of the one or more reprogramming factors has already begun. In other embodiments the plurality of MLPSCs are administered to the subject before expression of the one or more reprogramming factors has begun. In other embodiments the plurality of MLPSCs are administered at the same time as initiating reprogramming factor expression.

In some embodiments of any of the foregoing methods, the method also includes the step of delivering the one or more exogenous nucleic acids to the population of target cells.

In some embodiments of any of the foregoing methods the method also includes administering prostaglandin E2 to the subject. In some embodiments the method also includes administering a blocking antibody to the Natural Killer cell receptor NKG2D.

In a related aspect provided herein is a method for enhancing partial reprogramming of target cells in a post-natal subject in need thereof, the method comprising administering a plurality of MLPSCs, exosomes derived therefrom, or conditioned media derived from culture of a plurality of MLPSCs to a subject that expresses, concomitantly with the administration or after the administration, exogenous reprogramming factors comprising OCT4, SOX2, KLF4, and c-MYC in a population of target cells, wherein one or more exogenous nucleic acids for expression of the exogenous reprogramming factors have been delivered to the target cell population (i) as one or more synthetic mRNAs; (ii) by one or more non-integrating recombinant viruses; or (iii) as a combination of (i) and (ii); whereby a plurality of the target cells in the subject become partially reprogrammed.

In some embodiments the one or more exogenous nucleic acids are delivered to the target cell population as one or more synthetic mRNAs.

In other embodiments the one or more exogenous nucleic acids are delivered to the target cell population by one or more non-integrating recombinant viruses. In some embodiments the non-integrating recombinant virus is selected from the group consisting of: adenovirus, adeno-associated virus, non-integrating lentivirus, human cytomegalovirus (CMV), herpes simplex virus (HSV), and Sendai virus.

In some embodiments the MLPSCs are administered concomitantly with expression of the exogenous reprogramming factors. In other embodiments the MLPSCs are administered prior to expression of the exogenous reprogramming factors.

In some embodiments the method also includes delivering to the target cell population the one or more exogenous nucleic acids for expression of the exogenous reprogramming factors.

In some embodiments the MLPSCs are pre-licensed MLPSCs. In some embodiments, where the MLPSCs are pre-licensed MLPSCs, the pre-licensed MLPSCs are MLPSCs that were culture expanded in media containing: IFN-gamma and/or TNF-alpha; and one or more pro-inflammatory cytokines selected from the group consisting of IL-6, IL-8, IL-17A, MCP-1-alpha, MIP-1-beta, and IP-10. In some embodiments the media contains serum that contains IFN-gamma and/or TNF-alpha; and the one or more of the above-mentioned pro-inflammatory cytokines. In some embodiments, where the culture media contains serum, the culture medium contains newborn mammalian serum. In some embodiments the newborn mammalian serum is newborn calf serum.

In a related aspect provided herein is a population of MLPSCs when used in any of the foregoing methods as disclosed herein.

In a further aspect provided herein is a composition for enhancing partial reprogramming of target cells, comprising a plurality of modified mesenchymal lineage progenitor or stem cells (MLPSCs) comprising one or more exogenous nucleic acids encoding one or more: (a) reprogramming factors; or (b) targeted transactivators that induce expression of one or more endogenous reprogramming factors, wherein: (i) the modified MLPSCs, when in the presence of one or more target cells, deliver the one or more exogenous nucleic acids to the one more target cells; (ii) the one or more encoded reprogramming factors are expressed in the one or more target cells at a level sufficient to partially reprogram the one or more target cells; and (iii) the one or more encoded reprogramming factors or targeted transactivators are substantially inoperable to expression in the modified MLPSCs.

In a further aspect provided herein is a composition or kit for enhanced partial reprogramming of target cells, comprising:
(i) a culture-expanded population of human mesenchymal stem cells (hMSCs);
(ii) a conditionally-replicating helper virus; and
(iii) one or more helper-dependent viruses that: (a) comprise one or more expression cassettes for one or more reprogramming factors or targeted transactivators that induce expression of one or more endogenous reprogramming factors; and (b) substantially free of viral coding sequences.

In some embodiments the hMSCs are transduced with (ii), (iii), or both (ii) and (iii). In some embodiments the one or more expression cassettes are inducible and reversible expression cassettes. In some embodiments the conditionally replicating helper virus or the one or more helper-dependent viruses encode a regulatable transactivator (e.g., rtTA) that controls expression from the one or more inducible and reversible expression cassettes.

In some embodiments the conditionally replicating helper virus and the one or more helper-dependent viruses are adenoviruses, herpes simplex viruses (HSVs), non-integrating lentiviruses, or adeno-associated viruses (AAV). In some embodiments the conditionally-replicating helper virus and the one or more helper-dependent viruses are adenoviruses.

In some embodiments the culture-expanded hMSCs are pre-licensed hMSCs.

In some embodiments the composition is provided as a pharmaceutical composition comprising a pharmaceutically acceptable excipient. In some embodiments the pharmaceutical composition also includes one or more of prostaglandin E2 or a blocking antibody to the Natural Killer cell receptor NKG2D.

In a related aspect provided herein is a method for enhanced partial reprogramming of target cells comprising administering any of the just-mentioned compositions comprising the conditionally replicating virus and the helper-dependent virus to a subject in need thereof. In some embodiments the subject is suffering from or at risk of a health condition selected from the group consisting of: cardiovascular diseases, vascular endothelial conditions, low back pain, inflammatory diseases, osteoarthritis, metabolic diseases, kidney diseases, liver diseases, and neurological disorders. In some embodiments the transduced hMSCs are administered by a local route of administration.

### BRIEF DESCRIPTION OF DRAWINGS

FIG. 1: Serum cytokine levels assessment and comparison. 1:1 FCS/NBCS (serum A); fetal bovine serum (serum B); FBS from a different supplier (serum C). Panels A-I show measurements for cytokines IFN-gamma, IL-6, IL-8, and IL-17A, MCP-1, MIP-1 alpha, MIF-lbeta, IP-10, and TNF-alpha. respectively.

FIG. 2: Analysis of Change from Baseline at 12 Months in cardiac Echo Parameters—all subjects using pre-licensed MPCs cultured in media containing 10% FCS vs 5% FCS/5% NBCS.

FIG. 3: Analysis of Change from Baseline at 12 Months in Echo Parameters—subjects with persistent inflammation (hsCRP>2).

FIG. 4: Analysis of Change from Baseline at 12 Months in Echo Parameters—subjects without persistent inflammation (hsCRP<2).

FIG. 5: CV Death in in subjects with persistent inflammation (hsCRP>2) by MPCs cultured in the presence or absence of newborn serum.

FIG. 6: 3-Point composite MACE (MI, Stroke or CV Death) in subjects with persistent inflammation (hsCRP>2) by MPCs cultured in the presence or absence of newborn serum.

FIG. 7: 3-Point composite MACE (MI, Stroke or CV Death) in subjects administered MPCs cultured in the presence or absence of newborn serum in all or final passages. LHS figure represents data from all patients (A). RHS figure represents data from patients with persistent inflammation (CRP>2 mg/ml; (B)).

FIG. 8: 3-Point composite MACE (A) and Terminal Cardiac Events (TCE; B) in subjects with most severe disease (NTpro-BNP>1000 ng/ml; CRP>2 mg/ml) administered MPCs cultured in the presence or absence of newborn serum in all or final passages.

FIG. 9: Schematic Illustration of a combinatorial virus reprogramming platform in hMSCs.

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each embodiment described herein is to be applied mutatis mutandis to each and every other embodiment unless specifically stated otherwise.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purpose of exemplification only. Functionally-equivalent products, compositions and methods are clearly within the scope of the invention, as described herein.

The present invention is performed without undue experimentation using, unless otherwise indicated, conventional techniques of molecular biology, microbiology, virology, recombinant DNA technology, peptide synthesis in solution, solid phase peptide synthesis, and immunology. Such procedures are described, for example, in Sambrook, Fritsch & Maniatis, Molecular Cloning: A Laboratory Manual, Cold Spring Harbor Laboratories, New York, Second Edition (1989), whole of VoIs I, II, and DI; DNA Cloning: A Practical Approach, Vols. I and II (D. N. Glover, ed., 1985), IRL Press, Oxford, whole of text; Oligonucleotide Synthesis: A Practical Approach (M. J. Gait, ed, 1984) IRL Press, Oxford, whole of text, and particularly the papers therein by Gait, ppl-22; Atkinson et al, pp 35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984); Methods In Enzymology (S. Colowick and N. Kaplan, eds., Academic Press, Inc.), whole of series; J. F. Ramalho Ortigao, “The Chemistry of Peptide Synthesis” In: Knowledge database of Access to Virtual Laboratory website (Interactiva, Germany); Sakakibara, D., Teichman, J., Lien, E. Land Fenichel, R. L. (1976). Biochem. Biophys. Res. Commun. 73 336-342; Merrifield, R. B. (1963). J. Am. Chem. Soc. 85, 2149-2154; Barany, G. and Merrifield, R. B. (1979) in The Peptides (Gross, E. and Meienhofer, J. eds.), vol. 2, pp. 1-284, Academic Press, New York. 12. Wunsch, E., ed. (1974) Synthese von Peptiden in Houben-Weyls Metoden der Organischen Chemie (Müler, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474; Handbook of Experimental Immunology, VoIs. I-IV (D. M. Weir and C. C. Blackwell, eds., 1986, Blackwell Scientific Publications); and Animal Cell Culture: Practical Approach, Third Edition (John R. W. Masters, ed., 2000), ISBN 0199637970, whole of text.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

As used herein the term “derived from” shall be taken to indicate that a specified integer may be obtained from a particular source albeit not necessarily directly from that source.

As used herein, the term “prevent” or “preventing” or “prevention” shall be taken to mean administering a prophylactically effective amount of cells and stopping or hindering or delaying or reducing the development of at least one symptom of a clinical condition.

As used herein, the term “partial reprogramming” refers to a process whereby somatic cells are converted, by expression of one or more reprogramming factors for a sufficient period of time into an epigenetic state of a cell (i.e., a “partially reprogrammed cell”) retaining its original cell type or lineage identity, but exhibiting one or more alterations indicating a younger or “rejuvenated” cell age (commonly referred to as the “epigenetic age” or “eAge”) as indicated by changes in one or more parameters including, but not limited to, genomic methylation, gene expression profiles, metabolite production, and telomere length. For clarity, partially reprogrammed cells do not include cells reprogrammed to pluripotency or that have otherwise lost their original cell lineage. A sufficient period of time for expression of one or more reprogramming factors refers to a cumulative expression period that yields as a final outcome of the process partially reprogrammed cells that are substantially free of fully reprogrammed cells. In some embodiments the cumulative reprogramming factor expression period does not exceed about 21 days, preferably not more than 15 days.

As used herein, the term “full reprogramming” or “fully reprogrammed” refers to a process whereby one or more reprogramming factors are expressed for a period of time of a length sufficient to lose cells identity. Typically, full reprogramming requires a cumulative reprogramming factor expression period of longer than about 21 days.

As used herein, the phrase “STRO-1⁺ multipotential cells” shall be taken to mean non-hematopoietic STRO-1⁺ and/or TNAP⁺ progenitor cells capable of forming multipotential cell colonies. Preferred STRO-1⁺ multipotential cells are discussed in more detail herein.

As used herein, the term “conditioned media” as used in the context of the present disclosure refers to media obtained from MLPSCs under culture conditions. Such media contains the MLPSC secretome, proteins shed from the surface of MLPSCs, released metabolites, and particles such as extracellular vesicles. Accordingly, conditioned media of the disclosure may contain pro-angiogenic factors such as extracellular vesicles and Angiogenin or secreted metabolites such as prostaglandin E₂. In certain examples, the present disclosure relates to extracellular vesicles such as exosomes that have been obtained from conditioned media obtained from MLPSCs under culture conditions.

As used herein, the term “subject” shall be taken to mean any subject, preferably a mammal, e.g., mouse, rat, sheep, non-human primate, and human. In some preferred embodiments the subject is a human.

As used herein, the term “treat” or “treatment” or “treating” shall be understood to mean administering a therapeutically effective amount of cells and reducing or inhibiting at least one symptom of a clinical condition.

As used herein, the phrase “at risk of developing” a disease indicates that one or more clinical and/or diagnostic indicia are available that point to a higher than average probability of developing a particular health condition or disease.

### Methods

Provided herein is a method for enhancing partial reprogramming of target cells in a subject in need thereof, e.g., a human subject, where the method includes the step of administering a plurality of mesenchymal lineage progenitor or stem cells (MLPSCs), exosomes derived therefrom, or conditioned media derived from culture of MLPSCs to a subject that expresses or will express one or more reprogramming factors in a population of target cells, whereby, following a period of expression of the one or more reprogramming factors, a plurality of the target cells in the subject become partially reprogrammed, wherein the expression period is not of sufficient length to fully reprogram the plurality of target cells.

In some embodiments the target cells in the population of target cells are one or more of cardiomyocytes, nucleus pulposus cells, renal cells, liver cells, endothelial progenitors, chondrogenic progenitors, chondrocytes, neurons, and glial cells. In other embodiments the target cells comprise skin cells, e.g., keratinocytes, melanocytes, and fibroblasts.

In some embodiments the population of target cells comprises one or more exogenous nucleic acids encoding the one or more reprogramming factors. In other embodiments the population of target cells comprises one or more exogenous nucleic acids encoding one or more targeted transactivators for inducing expression of endogenous genes encoding the one or more reprogramming factors. In some embodiments the methods disclosed herein also encompass the step of delivering to a population of target cells in a subject the one or more exogenous nucleic acids. In some preferred embodiments the present methods do not encompass performance of the just-mentioned delivery step itself.

In some embodiments the MLPSCs are cultured expanded from a population of human STRO-1+ mesenchymal progenitor cells (MPCs). In other embodiments the MLPCs are culture-expanded human mesenchymal stem cells (hMSCs).

In some embodiments the MLPSCs to be administered are pre-licensed MLPSCs, which refers to MLPSCs culture-expanded in the presence of one or more pro-inflammatory stimuli, which is believed to enhance the anti-inflammatory characteristics of such MLPSCs, which is advantageous for their use in the methods described herein that include their administration to a subject in need thereof. In some embodiments MLPCS are pre-licensed by culture expansion in media containing IFN-gamma and/or TNF-alpha; and one or more pro-inflammatory cytokines selected from the group consisting of IL-6, IL-8, IL-17A, MCP-1-alpha, MIP-1-beta, and IP-10. In some embodiments the media used for culture expansion and pre-licensing of MLPSCs contains serum containing IFN-gamma and/or TNF-alpha; and the one or more of the foregoing pro-inflammatory cytokines. In some embodiments the culture media contains newborn mammalian serum. In some embodiments the newborn mammalian serum is newborn calf serum. In some embodiments the culture media contains a mixture of fetal mammalian serum and newborn mammalian serum. In some examples, the culture medium contains 5% (v/v) fetal calf serum and 5% (v/v) newborn calf serum. In other examples the culture medium contains newborn mammalian serum in the range of about 5% to about 10% (v/v), e.g., 6%, 7%, 8%, 9% or another percent from about 5% to about 10% (v/v) newborn mammalian serum.

In some embodiments the MLPSCs to be administered are genetically modified MLPSCs genetically modified to overexpress indoleamine 2,3-dioxygenase (IDO) and/or cyclooxygenase-2 (COX-2), the activity of each of which is believed to useful in suppressing NK cell cytotoxic activities believed to be detrimental to partial reprogramming of target cells in vivo.

In some embodiments MLPSCs are to be administered. In other embodiments isolated exosomes derived from MLPSCs are to be administered. In further embodiments conditioned media from culture of MLPSCs (e.g., culture of pre-licensed MLPSCs) is to be administered.

Generally, a subject in need of partial reprogramming of a population of target cells is subject suffering from a health condition to be treated by partial reprogramming of one or more target cell populations. In some embodiments the subject is suffering from a health condition selected from the group consisting of: cardiovascular diseases, vascular endothelial conditions, low back pain, inflammatory diseases, osteoarthritis, metabolic diseases, kidney diseases, liver diseases, and neurological disorders. In some preferred embodiments the subject is a human subject. In preferred embodiments the subject is a post-natal human subject. In some embodiments the age of the post-natal human subject is about 40 years to about 110 years, e.g., 45, 50, 55, 60, 65, 70, 75, 80, 85, 90, 95, 100, or another age from about 40 years to about 110 years.

In some embodiments administration of MLPCs, exosomes, or conditioned media is by a systemic route of administration (intravenous or intraarterial). In other embodiments administration is by a local route of administration preferably guided by the target tissue in which partial reprogramming is to be induced, e.g., intracardiac 5 administration for the heart, intra-articular administration for joints, intraverterbral administration for intravertebral disc, intracerebral for brain, etc.

### Reprogramming Factors

Suitable reprogramming factors for use in the partial reprogramming methods disclosed herein include, but are not limited to one or more of, OCT4, SOX2, KLF4, c-MYC, LIN28, and NANOG. In some embodiments the reprogramming factors comprise OCT4, SOX2, and KLF4. In other embodiments the reprogramming factors comprise OCT4, SOX2, KLF4, and c-MYC. In some embodiments the one or more reprogramming factors comprise OCT4, SOX2, and KLF4, but not c-MYC. In some embodiments the one or more reprogramming factors: (i) do not comprise LIN 28; or (ii) do not comprise NANOG.

In some embodiments the one or more reprogramming factors consist of: (i) OCT4, SOX2, KLF4, c-MYC, LIN28, and NANOG; (ii) OCT4, SOX2, KLF4, and c-MYC; or (iii) OCT4, SOX2, and KLF4.

In some preferred embodiments the reprogramming factors comprise OCT4, SOX2, KLF4, and c-MYC.

Also contemplated herein is the use of structural variants of any of the above-mentioned reprogramming factors (e.g., fusion proteins, truncations, point mutations, or deletions) that have been functionally tested for cell reprogramming to pluripotency in vitro. Examples of reprogramming factor fusion proteins, e.g., OCT4-VP16 fusion proteins are known in the art, e.g., Hammachi et al., (2012), Cell Reports, 1(2):99-109.

In some embodiments the one or more reprogramming factors are fusion proteins comprising a conditional destabilizing domain (CDD), whereby in the absence of a synthetic stabilising ligand, the CDD triggers rapid degradation of the fusion protein. This system allows fine tuning of reprogramming factor-fusion protein levels by controlling the level of the synthetic stabilising ligand present. In some exemplary embodiments the CDD is a dihydrofolate reductase CDD and the synthetic stabilising ligand is trimethoprim. Examples of suitable reprogramming factor fusion proteins are known in the art, e.g., in Sui et al., (2014), Stem Cell Reports, 2(5):721-733.

Targeted transactivators for inducing expression of any of the above-mentioned reprogramming factors from the corresponding endogenous/native genes have been described in the art. In some preferred embodiments the targeted transactivators are CRISPR activation (CRISPRa)-based targeted transactivators. CRISPR activation (CRISPRa) uses a catalytically inactivated form of Cas9 (dCas9) fused with a transactivator domain that enables the activation of transcription from endogenous promoters. Generally a CRISPRa-based targeted transactivator will comprise two components: (i) a dCas9-transactivator domain fusion protein-“dCas9-tA” (e.g., a VP16 transactivation domain); and (ii) a guide RNA (gRNA) that directs the dCas9-tA to a specific reprogramming factor gene locus in the genome to drive expression of the endogenously encoded reprogramming factor. Suitable dCas9-tAs and reprogramming factor gRNAs, and methods for using targeted transactivators for cell reprogramming are known in the art, as described in, e.g., Weltner et al., (2018), Nat Commun; 9:2643 and Sokka et al., (2022), Stem Cell Reports, 17(2):413-426.

In some embodiments the one or more exogenous nucleic acids include one or more synthetic mRNAs encoding the one or more reprogramming factors or encoding the one or more targeted transactivators. In some embodiments the one or more synthetic mRNAs comprise one or more nucleoside-modified mRNAs, where the nucleoside modified mRNAs are RNASe-resistant and/or less likely to induce a Type I interferon response. Methods for in vitro production of mRNAs for reprogramming are known in the art as reviewed in, e.g., Steinle et al. (2016), Stem Cells, 35(1):68-79. In some embodiments, where synthetic mRNAs are used, the synthetic mRNAs comprise both a mixture of both nucleoside-modified mRNAs and non-nucleoside modified mRNAs. Kits for in vitro transcription (IVT) of modified or unmodified mRNAs are commercially available, e.g., the HyperScribe™ All in One mRNA Synthesis Kits from APExBIO (Cat. Nos:K1063-K1068).

Methods and compositions for in vivo delivery of mRNAs systemically or to particular tissues are known in the art as reviewed in Zhang et al., (2021), Chemical Reviews, 121:12181-12277. In some embodiments lipid nanoparticles (LNPs) are used for delivery of mRNAs to a subject in the methods disclosed herein. In other embodiments mRNAs are delivered by polyplex nanomicelles as described in, e.g., Chang et al., (2022), International Journal of Molecular Sciences, 23:565. In other embodiments, particularly where mRNAs are to be administered directly to a tissue comprising the target cells of interest (e.g., in the heart), “naked” mRNA, i.e., mRNA not associated with a transfection agent, is injected in biocompatible buffer formulation (e.g., citrate-saline solution), as exemplified by Carlsson et al., (2018), Molecular TherapyMethods & Clinical Development, 9:330-346.

In some embodiments the one or more nucleic acids comprise one or more non-integrated expression cassettes for expression of the one or more encoded reprogramming factors or targeted transactivators in mammalian cells (e.g., a population of target cells in a human subject), where an expression cassette, at a minimum, includes a promoter operably linked to an open reading frame encoding one or more reprogramming factors, a targeting enzyme (e.g., dCas9), or a gRNA targeting the promoter of a native gene encoding a reprogramming factor.

As used herein, the term “promoter” is to be taken in its broadest context and includes the transcriptional regulatory sequences of a genomic gene, including the TATA box or initiator element, which is required for transcription initiation, with or without additional regulatory elements (i.e., upstream activating sequences, transcription factor binding sites, enhancers and silencers) which alter gene expression, e.g., in response to developmental and/or external stimuli, or in a tissue specific manner. In the present context, the term “promoter” is also used to describe a recombinant, synthetic or fusion molecule, or derivative which confers, activates or enhances the expression of a nucleic acid to which it is operably-linked, and preferably which encodes a peptide or protein. Preferred promoters can contain additional copies of one or more specific regulatory elements to further enhance expression and/or alter the spatial expression and/or temporal expression of said nucleic acid molecule.

In the present context, a nucleic acid is “operably-linked” with or to a promoter (i.e., under the regulatory control of a promoter) when it is positioned such that its expression is controlled by the promoter. Promoters are generally positioned 5′ (upstream) to the nucleic acid, the expression of which they control. To construct heterologous promoter/nucleic acid combinations, it is generally preferred to position the promoter at a distance from the gene transcription start site that is approximately the same as the distance between that promoter and the gene it controls in its natural setting, i.e., the gene from which the promoter is derived. As is known in the art, some variation in this distance can be accommodated without loss of promoter function. Similarly, the preferred positioning of a regulatory sequence element with respect to a heterologous nucleic acid to be placed under its control is defined by the positioning of the element in its natural setting, i.e., the gene from which it is derived. Again, as is known in the art, some variation in this distance can also occur.

In some embodiments the one or more non-integrated expression cassettes are provided as one or more: plasmid expression vectors, minicircle expression vectors, linear amplicons, circular DNA amplicons, recombinant DNA viral genomes or amplicons, recombinant RNA viral genomes, RNA amplicons, or any combinations thereof.

In some preferred embodiments, plasmid expression vectors, minicircle expression vectors, linear amplicons, and circular DNA amplicons are provided as episomal vectors, which contain an origin of replication whereby an episomal vector is replicated when its host cell undergoes cell division. Episomal vectors are particularly useful when a population of target cells to be partially reprogrammed is proliferative (e.g., chondrocytes, glial cells, and neural progenitors). The use of episomal expression vector for reprogramming is known in the art, e.g., in Wang and Loh (2019), Cell Transplant, 28(1 Suppl):112S-131S, and episomal vectors.

In some embodiments non-integrative expression cassettes are delivered by a recombinant virus selected from the group consisting of adenovirus, adeno-associated virus, non-integrating lentivirus, human cytomegalovirus (CMV), herpes simplex virus (HSV), and Sendai virus. While not wishing to be bound by theory, it is believed that administration of hMLSCs shortly before or after administration of a recombinant (non-integrative) virus has the added advantage of reducing unwanted immunoneutralization of the such recombinant viruses. This reduced immunoneutralization leads to increased viral transduction of target cells and, ultimately, increased partial reprogramming efficiency in the target cells.

In some embodiments, where the one or more expression cassettes are provided as one or more DNA viral genomes, the DNA viral genomes are provided as recombinant non-integrating lentivirus, which have very high transduction efficiency while minimising risk of insertional mutagenesis. See, e.g., Gurumoorthy et al., (2022), Biomedicines, 10:107 and Abouleisa et al., (2022), Circulation, 145:1339-1355.

In other embodiments one or more recombinant adenoviruses provide the one or more expression cassettes as described in, e.g., Lehmann et al., (2019), Gene Therapy, 26:432-440.

In other embodiments one or more recombinant human cytomegaloviruses (hCMV), which have the advantage of being able to accommodate very large inserts relative to other viral vectors, are used in the methods disclosed herein. The design us use of recombinant CMV as a gene therapy vector has been described in the art, e.g., in Jaiyan et al., (2022), Proc. Natl. Acad. Sci USA, 119(20):e2121499119.

In some embodiments where the one or more expression cassettes are provided as one or more RNA viral genomes, the RNA viral genomes are provided as recombinant RNA viruses, which essentially present no risk of insertional mutagenesis. In some embodiments the recombinant RNA virus to be used is a measles virus (MV), a human negative strand RNA Paramyxovirus that has a proven safety vaccination record. The use of MV for reprogramming has been described in, e.g., Wang et al., (2019), 26(5):151-164.

In some embodiments, where a recombinant virus is utilised, the recombinant virus is a recombinant virus have a target cell-selective tropism. For example, recombinant adeno-associated viruses (AAVs) engineered to have tropism for specific target cell populations after systemic administration, e.g., for cardiomyocytes (Zincarelli et al., 2008, Mol. Ther., 16(6):1073-1080) and CNS neurons and/or astrocytes (Goertsen et al., 2021, Nature Neuroscience, 25:106-115) have been developed in the art.

In some embodiments the promoter in an expression cassette is a target cell-selective promoter. In some embodiments the target cell-selective promoter is one selected from among: cardiomyocyte-selective promoters, nucleus pulposus cell-selective promoters, renal cell-selective promoters, liver cell-selective promoters, endothelial progenitor-selective promoters, chondrocyte-selective promoters, glial cell-selective promoters, neural progenitor-selective promoters, and neuron-selective promoters. Suitable cardiomyocyte-selective promoters include, but are not limited to: the cardiac-specific troponin T promoter (TNNT2), atrial natriuretic factor (ANF) promoter, and the Na⁺—Ca⁺ exchanger (NCX1) promoter. An exemplary nucleus pulposus-selective promoter is the carbonic anhydrase 12 (CA12) promoter. Exemplary kidney-selective promoters include, kidney-specific cadherin (KSPC) Na/glucose co-transporter (SGLT2) gene promoter in proximal tubule, and 2 chloride co-transporter (NKCC2) gene promoter for expression in the thick ascending limb of Henle's loop. An exemplary chondrocyte-selective promoter is the collagen type II (COL2A1) promoter. Exemplary liver-selective promoters are the serum albumin gene promoter and the alpha-1-antitrypsin gene promoter. Exemplary glial-selective promoters include glial fibrillary acidic protein GFAP gene promoter for astrocytes and the S1000 gene promoter for astrocytes and myelinating oligodendrocytes. Exemplary neuron-selective promoters include: CamKII gene promoter and synapsin gene promoter.

In some embodiments, where a gRNA is to be expressed as part of a targeted transactivator, a suitable promoter for expression of the gRNA is a PolIII promoter such as a U3 or U6 promoter. In other embodiments expression of both both a gRNA and the targeting enzyme (e.g., dCas9), i.e., both components of a targeted transactivator, can be driven by a single promoter, e.g., the H1 promoter, which can be utilised by both RNA polymerase II and III (Gao et al., 2018, Mol. Ther. Nucleic Acids, 14:32-40).

In some embodiments the promoter in an expression cassette is an inducible (and reversible) promoter, referred to as simply an “inducible promoter.” Generally, an inducible promoter as referred to herein is a promoter the activity of which is under the control of an exogenously provided inducible transactivator than can be conditionally activated or inactivated. In some embodiments the transactivator is a ligand-modulated or photo-modulated transactivator capable of transactivating the inducible promoter under expression-permissive conditions. Suitable inducible transactivators include, but are not limited to reverse tetracycline transactivators (rtTAs), tetracycline (off) transactivators (tTas), cumate-inducible transactivators, FK506-inducible transactivators, and red light/far red light inducible-REDMAP transactivators (see, e.g., Zhou et al., 2022, Nature Biotechnology, 40(2):262-272). Inducible promoters and transactivation systems are particularly useful in the context of partial reprogramming, as their use permits well controlled timing of reprogramming factor expression in a subjected to be treated according to the methods disclosed herein.

In some embodiments at least one of the exogenous nucleic acids provided to a subject to be treated is polycistronic, i.e., it encodes two or more proteins (e.g., reprogramming factors). In some embodiments, a polycistronic synthetic mRNA is provided. In other embodiments the provided nucleic acid comprises a polycistronic expression cassette encoding a polycistronic mRNA (a “polycistronic expression cassette”), which mRNA, upon translation gives rise to independent polypeptides comprising different amino acid sequences or functionalities. In some embodiments, a polycistronic expression cassette encodes a “polyprotein” comprising multiple polypeptide sequences that are separated by encoded by a picornavirus, e.g., a foot-and-mouth disease virus (FMDV) viral 2A peptide sequence. The 2A peptide sequence acts co-translationally, by preventing the formation of a normal peptide bond between the conserved glycine and last proline, resulting in ribosome skipping to the next codon, and the nascent peptide cleaving between the Gly and Pro. After cleavage, the short 2A peptide remains fused to the C-terminus of the “upstream” protein, while the proline is added to the N-terminus of the “downstream” protein, which during translation allow cleavage of the nascent polypeptide sequence into separate polypeptides (see, e.g., Trichas et al., (2008), BMC Biology 6:40.

In other embodiments, a polycistronic mRNA or expression cassette incorporates one or more internal ribosomal entry site (IRES) sequences. IRES sequences and their use are known in the art as exemplified in, e.g., Martinez-Sales, (1999), Current Opinion in Biotechnology, 10:458-464.

In some embodiments the one or more exogenous nucleic acids use a combination of 2A and IRES elements in a contiguous nucleic acid. In some embodiments, a single expression cassette or synthetic mRNA encodes four reprogramming factors (RFs) in the following configuration: RF1-2a-RF2-IRES-RF3-2a-RF4. Such a configuration is exemplified in, e.g., Lehmann et al., (2019), Current Gene Therapy, 19(4):248-254, in which the polycistronic expression cassette OCT4-2a-KLF4-IRES-SOX2-2a-c-MYC was used for partial reprogramming. In other embodiments, two separate expression cassettes are provided as RF1-2a-RF2 and RF3-2a-RF4.

Accordingly also provided herein is a method for method for enhancing partial reprogramming of target cells in a post-natal subject in need thereof, the method comprising administering a plurality of mesenchymal lineage progenitor or stem cells (MLPSCs), exosomes derived therefrom, or conditioned media derived from culture of the plurality of a plurality MLPSCs to a subject that expresses, concomitantly with the administration or after the administration, exogenous reprogramming factors comprising OCT4, SOX2, KLF4, and c-MYC in a population of target cells, wherein one or more exogenous nucleic acids for expression of the exogenous reprogramming factors have been delivered to the target cell population (i) as one or more synthetic mRNAs; (ii) by one or more non-integrating recombinant viruses; or (iii) as a combination of (i) and (ii); whereby a plurality of the target cells in the post-natal subject become partially reprogrammed.

Also provided is a composition for enhancing partial reprogramming of target cells, comprising a plurality of modified mesenchymal lineage progenitor or stem cells (MLPSCs) comprising one or more exogenous nucleic acids encoding one or more: (a) reprogramming factors; or (b) targeted transactivators that induce expression of one or more endogenous reprogramming factors, wherein: (i) the modified MLPSCs, when in the presence of one or more target cells, deliver the one or more exogenous nucleic acids to the one more target cells; (ii) the one or more encoded reprogramming factors are expressed in the one or more target cells at a level sufficient to partially reprogram the one or more target cells; and (iii) the one or more encoded reprogramming factors or targeted transactivators are substantially inoperable to expression in the modified MLPSCs prior to the delivery in (i).

Also disclosed herein is a composition for enhanced partial reprogramming of target cells, comprising:
(i) a culture-expanded population of human mesenchymal stem cells (hMSCs); (ii) a conditionally-replicating helper virus; and (iii) one or more helper-dependent viruses that: (a) comprise one or more expression cassettes for one or more reprogramming factors or targeted transactivators that induce expression of one or more endogenous reprogramming factors; and (b) substantially free of viral coding sequences. In some preferred embodiments the conditionally replicating helper virus and the helper-dependent reprogramming virus are adenoviruses. Conditionally replicating helper and helper-dependent adenoviruses are known to replicate at sufficient level in hMSCs to expand in and be secreted so as to diffuse to nearby cells. See Mckenna et al., 2021, Mol. Therapy, 29(5):1808-1820. In some embodiments the helper and helper-dependent adenoviruses are both serotype 5 adenoviruses. It is believed that using the hMSCs for reprogramming viruses may shield such viruses from immunoneutralisation during partial reprogramming in vivo. In some embodiments such compositions also include or more of prostaglandin E2 or a blocking antibody to the Natural Killer cell receptor NKG2D. In some embodiments the foregoing compositions are used in a method for enhanced partial reprogramming in vivo.

In preferred embodiments of the partial reprogramming methods disclosed herein the one or more reprogramming factors as disclosed herein are inducibly and reversibly expressed for a period of at least two consecutive days, but not for more than about 21 consecutive days, e.g., 3 days, 4 days, 5 days, 6 days, 7 days, 8 days, 9 days, 10 days, 11 days, 12 days, 14 days, 15 days, 16 days, 18 days, 20 days, or another number of consecutive days from at least two consecutive days to 21 consecutive days. Limiting the expression period of reprogramming factors in the population of target cells reduces the possibility of going beyond partial reprogramming to full reprogramming and loss of target cell identity, which is undesirable. In some preferred embodiments the one or more reprogramming factors are inducibly and reversibly expressed for a period of at least two consecutive days, but not for more than 15 days.

In some embodiments the one or more reprogramming factors are expressed for a cumulative period of at least two days, but not longer than about 21 days, e.g., 3 days, 4 days, 5 days, 6 days, 7 days, 8 days, 9 days, 10 days, 11 days, 12 days, 14 days, 15 days, 16 days, 18 days, 20 days, or another cumulative period of at least two days but not more than 21 days. For example, the one or more reprogramming factors can be expressed for a first period of seven days, followed by little or no expression of about seven days, followed by a second period of expression of the one or more reprogramming factors of about seven days before ending expression of the one or more reprogramming factors.

### Mesenchymal Lineage Precursor or Stem Cells

As used herein, the term “mesenchymal lineage precursor or stem cell (MLPSC)” refers to undifferentiated multipotent cells that have the capacity to self-renew while maintaining multipotency and the capacity to differentiate into a number of cell types either of mesenchymal origin, for example, osteoblasts, chondrocytes, adipocytes, stromal cells, fibroblasts and tendons, or non-mesodermal origin, for example, hepatocytes, neural cells and epithelial cells. For the avoidance of doubt, a “mesenchymal lineage precursor cell” refers to a cell which can differentiate into a mesenchymal cell such as bone, cartilage, muscle and fat cells, and fibrous connective tissue.

The term “mesenchymal lineage precursor or stem cells” includes both parent cells and their undifferentiated progeny. The term also includes mesenchymal precursor cells, multipotent stromal cells, mesenchymal stem cells (MSCs), perivascular mesenchymal precursor cells, and their undifferentiated progeny. Mesenchymal lineage precursor or stem cells can be autologous, allogeneic, xenogenic, syngenic or isogenic. Autologous cells are isolated from the same individual to which they will be reimplanted. Allogeneic cells are isolated from a donor of the same species. Xenogenic cells are isolated from a donor of another species. Syngenic or isogenic cells are isolated from genetically identical organisms, such as twins, clones, or highly inbred research animal models.

In an example, the mesenchymal lineage precursor or stem cells are allogeneic. In an example, the allogeneic mesenchymal lineage precursor or stem cells are culture expanded and cryopreserved.

Mesenchymal lineage precursor or stem cells reside primarily in the bone marrow, but have also shown to be present in diverse host tissues including, for example, cord blood and umbilical cord, adult peripheral blood, adipose tissue, trabecular bone and dental pulp. They are also found in skin, spleen, pancreas, brain, kidney, liver, heart, retina, brain, hair follicles, intestine, lung, lymph node, thymus, ligament, tendon, skeletal muscle, dermis, and periosteum; and are capable of differentiating into germ lines such as mesoderm and/or endoderm and/or ectoderm. Thus, mesenchymal lineage precursor or stem cells are capable of differentiating into a large number of cell types including, but not limited to, adipose, osseous, cartilaginous, elastic, muscular, and fibrous connective tissues. The specific lineage-commitment and differentiation pathway which these cells enter depends upon various influences from mechanical influences and/or endogenous bioactive factors, such as growth factors, cytokines, and/or local microenvironmental conditions established by host tissues. The terms “enriched”, “enrichment” or variations thereof are used herein to describe a population of cells in which the proportion of one particular cell type or the proportion of a number of particular cell types is increased when compared with an untreated population of the cells (e.g., cells in their native environment). In one example, a population enriched for mesenchymal lineage precursor or stem cells comprises at least about 0.1% or 0.5% or 1% or 2% or 5% or 10% or 15% or 20% or 25% or 30% or 50% or 75% mesenchymal lineage precursor or stem cells. In this regard, the term “population of cells enriched for mesenchymal lineage precursor or stem cells” will be taken to provide explicit support for the term “population of cells comprising X % mesenchymal lineage precursor or stem cells”, wherein X % is a percentage as recited herein. The mesenchymal lineage precursor or stem cells can, in some examples, form clonogenic colonies, e.g. CFU-F (fibroblasts) or a subset thereof (e.g., 50% or 60% or 70% or 70% or 90% or 95%) can have this activity.

In an example of the present disclosure, the mesenchymal lineage precursor or stem cells are mesenchymal stem cells (MSCs). The MSCs may be a homogeneous composition or may be a mixed cell population enriched in MSCs. Homogeneous MSC compositions may be obtained by culturing adherent marrow or periosteal cells, and the MSCs may be identified by specific cell surface markers which are identified with unique monoclonal antibodies. A method for obtaining a cell population enriched in MSCs is described, for example, in U.S. Pat. No. 5,486,359. Alternative sources for MSCs include, but are not limited to, blood, skin, cord blood, muscle, fat, bone, and perichondrium. In an example, the MSCs are allogeneic. In an example, the MSCs are cryopreserved. In an example, the MSCs are culture expanded and cryopreserved. In another example, the mesenchymal lineage precursor or stem cells are CD29+, CD54+, CD73+, CD90+, CD102+, CD105+, CD106+, CD166+, MHC1+MSCs.

In an example, the mesenchymal lineage precursor or stem cells are culture expanded from a population of MSCs that express markers, including CD73, CD90, CD105 and CD166, and lack expression of hematopoietic cell surface antigens such as CD45 and CD31. For example, the mesenchymal lineage precursor or stem cells can be culture expanded from a population of MSCs that are CD73+, CD90+, CD105+, CD166+, CD45− and CD31−. In an example, the population of MSCs is further characterized by low levels of major histocompatibility complex (MHC) class I. In another example, the MSCs are negative for major histocompatibility complex class II molecules, and are negative for costimulatory molecules CD40, CD80, and CD86. In an example, the culture expansion comprises 5 passages.

Isolated or enriched mesenchymal lineage precursor or stem cells can be expanded in vitro by culture. Isolated or enriched mesenchymal lineage precursor or stem cells can be cryopreserved, thawed and subsequently expanded in vitro by culture. In one example, isolated or enriched mesenchymal lineage precursor or stem cells are seeded at 50,000 viable cells/cm² in culture medium (serum free or serum-supplemented), for example, alpha minimum essential media (αMEM) supplemented with 5% fetal bovine serum (FBS) and glutamine, and allowed to adhere to the culture vessel overnight at 37° C., 20% O₂. The culture medium is subsequently replaced and/or altered as required and the cells cultured for a further 68 to 72 hours at 37° C., 5% O₂.

As will be appreciated by those of skill in the art, cultured mesenchymal lineage precursor or stem cells are phenotypically different to cells in vivo. For example, in one embodiment they express one or more of the following markers, CD44, NG2, DC146 and CD140b. Cultured mesenchymal lineage precursor or stem cells are also biologically different to cells in vivo, having a higher rate of proliferation compared to the largely non-cycling (quiescent) cells in vivo.

In one example, the population of cells is enriched from a cell preparation comprising STRO-1+ cells in a selectable form. In this regard, the term “selectable form” will be understood to mean that the cells express a marker (e.g., a cell surface marker) permitting selection of the STRO-1+ cells. The marker can be STRO-1, but need not be. For example, as described and/or exemplified herein, cells (e.g., mesenchymal precursor cells) expressing STRO-2 and/or STRO-3 (TNAP) and/or STRO-4 and/or VCAM-1 and/or CD146 and/or 3G5 also express STRO-1 (and can be STRO-lbright). Accordingly, an indication that cells are STRO-1+ does not mean that the cells are selected solely by STRO-1 expression. In one example, the cells are selected based on at least STRO-3 expression, e.g., they are STRO-3+(TNAP+). Reference to selection of a cell or population thereof does not necessarily require selection from a specific tissue source. As described herein STRO-1+ cells can be selected from or isolated from or enriched from a large variety of sources. That said, in some examples, these terms provide support for selection from any tissue comprising STRO-1+ cells (e.g., mesenchymal precursor cells) or vascularized tissue or tissue comprising pericytes (e.g., STRO-1+ pericytes) or any one or more of the tissues recited herein.

In one example, the cells used in the present disclosure express one or more markers individually or collectively selected from the group consisting of TNAP+, VCAM-1+, THY-1+, STRO-2+, STRO-4+(HSP-900), CD45+, CD146+, 3G5+ or any combination thereof.

By “individually” is meant that the disclosure encompasses the recited markers or groups of markers separately, and that, notwithstanding that individual markers or groups of markers may not be separately listed herein the accompanying claims may define such marker or groups of markers separately and divisibly from each other.

By “collectively” is meant that the disclosure encompasses any number or combination of the recited markers or groups of markers, and that, notwithstanding that such numbers or combinations of markers or groups of markers may not be specifically listed herein the accompanying claims may define such combinations or sub-combinations separately and divisibly from any other combination of markers or groups of markers.

As used herein the term “TNAP” is intended to encompass all isoforms of tissue non-specific alkaline phosphatase. For example, the term encompasses the liver isoform (LAP), the bone isoform (BAP) and the kidney isoform (KAP). In one example, the TNAP is BAP. In one example, TNAP as used herein refers to a molecule which can bind the STRO-3 antibody produced by the hybridoma cell line deposited with ATCC on 19 Dec. 2005 under the provisions of the Budapest Treaty under deposit accession number PTA-7282.

Furthermore, in one example, the STRO-1+ cells are capable of giving rise to clonogenic CFU-F.

In one example, a significant proportion of the STRO-1+ cells are capable of differentiation into at least two different germ lines. Non-limiting examples of the lineages to which the STRO-1+ cells may be committed include bone precursor cells; hepatocyte progenitors, which are multipotent for bile duct epithelial cells and hepatocytes; neural restricted cells, which can generate glial cell precursors that progress to oligodendrocytes and astrocytes; neuronal precursors that progress to neurons; precursors for cardiac muscle and cardiomyocytes, glucose-responsive insulin secreting pancreatic beta cell lines. Other lineages include, but are not limited to, odontoblasts, dentin-producing cells and chondrocytes, and precursor cells of the following: retinal pigment epithelial cells, fibroblasts, skin cells such as keratinocytes, dendritic cells, hair follicle cells, renal duct epithelial cells, smooth and skeletal muscle cells, testicular progenitors, vascular endothelial cells, tendon, ligament, cartilage, adipocyte, fibroblast, marrow stroma, cardiac muscle, smooth muscle, skeletal muscle, pericyte, vascular, epithelial, glial, neuronal, astrocyte and oligodendrocyte cells. In an example, mesenchymal lineage precursor or stem cells are obtained from a single donor, or multiple donors where the donor samples or mesenchymal lineage precursor or stem cells are subsequently pooled and then culture expanded.

MLPSCs encompassed by the present disclosure may also be cryopreserved prior to administration to a subject. In an example, mesenchymal lineage precursor or stem cells are culture expanded and cryopreserved prior to administration to a subject.

In an example, the present disclosure encompasses mesenchymal lineage precursor or stem cells as well as progeny thereof, soluble factors derived therefrom, and/or extracellular vesicles isolated therefrom. In another example, the present disclosure encompasses mesenchymal lineage precursor or stem cells as well as extracellular vesicles isolated therefrom. For example, it is possible to culture expand mesenchymal precursor lineage or stem cells of the disclosure for a period of time and under conditions suitable for secretion of extracellular vesicles into the cell culture medium. Secreted extracellular vesicles can subsequently be obtained from the culture medium for use in therapy.

The term “extracellular vesicles” as used herein, refers to lipid particles naturally released from cells and ranging in size from about 30 nm to as a large as 10 microns, although typically they are less than 200 nm in size. They can contain proteins, nucleic acids, lipids, metabolites, or organelles from the releasing cells (e.g., mesenchymal stem cells; STRO-1⁺ cells).

The term “exosomes” as used herein, refers to a type of extracellular vesicle generally ranging in size from about 30 nm to about 150 nm and originating in the endosomal compartment of mammalian cells from which they are trafficked to the cell membrane and released. They may contain nucleic acids (e.g., RNA; microRNAs), proteins, lipids, and metabolites and function in intercellular communication by being secreted from one cell and taken up by other cells to deliver their cargo.

MLPSCs can be found in bone marrow, blood, dental pulp cells, adipose tissue, skin, spleen, pancreas, brain, kidney, liver, heart, retina, brain, hair follicles, intestine, lung, lymph node, thymus, bone, ligament, tendon, skeletal muscle, dermis, and periosteum; and are capable of differentiating into germ lines such as mesoderm and/or endoderm and/or ectoderm. In some preferred embodiments, the MLPSCs are from bone marrow, dental pulp or adipose tissue, more preferably from dental pulp or adipose tissue. Thus, MLPSCs are capable of differentiating into a large number of cell types including, but not limited to, adipose, osseous, cartilaginous, elastic, muscular, and fibrous connective tissues. The specific lineage-commitment and differentiation pathway which these cells enter depends upon various influences from mechanical influences and/or endogenous bioactive factors, such as growth factors, cytokines, and/or local microenvironmental conditions established by host tissues. MLPSCs cells are thus non-hematopoietic progenitor cells which divide to yield daughter cells that are either stem cells or are precursor cells which in time will irreversibly differentiate to yield a phenotypic cell.

In a preferred embodiment, the MLPSCs cells to be used in the methods disclosed herein are enriched from a sample obtained from a subject, e.g., a subject to be treated or a related subject or an unrelated subject (whether of the same species or different). Such an enrichment may be performed ex vivo or in vitro The terms ‘enriched’, ‘enrichment’ or variations thereof are used herein to describe a population of cells in which the proportion of one particular cell type or the proportion of a number of particular cell types is increased when compared with the untreated population.

In some preferred embodiments, the cells used in the present invention are positive for expression of more markers individually or collectively selected from the group consisting of: STRO-1⁺, TNAP⁺, VCAM-1⁺, THY-1⁺, STRO-2⁺, CD146⁺, 3G5⁺, or any combination thereof. In some preferred embodiments the MLPSCs are enriched for STRO-1⁺ mesenchymal precursor cells (MPCs) or culture-expanded multipotential progeny thereof.

Preferably, the STRO-1⁺ cells are STRO-1^{bright} (syn. STRO-1^{bri}). Preferably, the STRO-1^{bright} cells are additionally one or more of TNAP⁺, VCAM-1⁺, THY-1⁺, STRO-2⁺ and/or CD146⁺. In one embodiment the MLPSCs are perivascular mesenchymal precursor cells as defined in WO 2004/85630.

In some embodiments, culture-expanded MLPSCs express one or more markers collectively or individually selected from the group consisting of TNF-R1, TGFβ1, LFA-3, THY-1, VCAM-1, ICAM-1, PECAM-1, P-selectin, L-selectin, 3G5, CD49a/CD49b/CD29, CD49c/CD29, CD49d/CD29, CD 90, CD29, CD18, CD61, integrin beta 6-19, thrombomodulin, CD10, CD13, SCF, PDGF-R, EGF-R, IGF1-R, NGF-R, FGF-R, Leptin-R (STRO-2=Leptin-R), RANKL, STRO-1, CD146 or any combination of these markers.

A cell that is referred to as being “positive” for a given marker it may express either a low (lo or dim) or a high (bright, bri) level of that marker depending on the degree to which the marker is present on the cell surface, where the terms relate to intensity of fluorescence or other marker used in the sorting process of the cells. The distinction of 1o (or dim or dull) and bri will be understood in the context of the marker used on a particular cell population being sorted. A cell that is referred to as being “negative” for a given marker is not necessarily completely absent from that cell. This term means that the marker is expressed at a relatively very low level by that cell, and that it generates a very low signal when detectably labelled or is undetectable above background levels.

The term “bright”, when used herein, refers to a marker on a cell surface that generates a relatively high signal when detectably labelled. Whilst not wishing to be limited by theory, it is proposed that “bright” cells express more of the target marker protein (for example the antigen recognised by STRO-1) than other cells in the sample. For instance, STRO-1^{bri} cells produce a greater fluorescent signal, when labelled with a FITC-conjugated STRO-1 antibody as determined by fluorescence activated cell sorting (FACS) analysis, than non-bright cells (STRO-1^{dull/dim}). Preferably, “bright” cells constitute at least about 0.1% of the most brightly labelled bone marrow mononuclear cells contained in the starting sample. In other embodiments, “bright” cells constitute at least about 0.1%, at least about 0.5%, at least about 1%, at least about 1.5%, or at least about 2%, of the most brightly labelled bone marrow mononuclear cells contained in the starting sample. In a preferred embodiment, STRO-1^{bright} cells have 2 log magnitude higher expression of STRO-1 surface expression relative to “background”, namely cells that are STRO-1⁻. By comparison, STRO-1^{dim} and/or STRO-1^{intermediate} cells have less than 2 log magnitude higher expression of STRO-1 surface expression, typically about 1 log or less than “background”.

As used herein the term “TNAP” is intended to encompass all isoforms of tissue non-specific alkaline phosphatase. For example, the term encompasses the liver isoform (LAP), the bone isoform (BAP) and the kidney isoform (KAP). In a preferred embodiment, the TNAP is BAP. In a particularly preferred embodiment, TNAP as used herein refers to a molecule which can bind the STRO-3 antibody produced by the hybridoma cell line deposited with ATCC on 19 Dec. 2005 under the provisions of the Budapest Treaty under deposit accession number PTA-7282.

Furthermore, in a preferred embodiment, the STRO-1+ multipotential cells are capable of giving rise to clonogenic CFU-F.

It is preferred that a significant proportion of the MLPSCs are capable of differentiation into at least two different germ lines. Non-limiting examples of the lineages to which the multipotential cells may be committed include bone precursor cells; hepatocyte progenitors, which are multipotent for bile duct epithelial cells and hepatocytes; neural restricted cells, which can generate glial cell precursors that progress to oligodendrocytes and astrocytes; neuronal precursors that progress to neurons; precursors for cardiac muscle and cardiomyocytes, glucose-responsive insulin secreting pancreatic beta cell lines. Other lineages include, but are not limited to, odontoblasts, dentin-producing cells and chondrocytes, and precursor cells of the following: retinal pigment epithelial cells, fibroblasts, skin cells such as keratinocytes, dendritic cells, hair follicle cells, renal duct epithelial cells, smooth and skeletal muscle cells, testicular progenitors, vascular endothelial cells, tendon, ligament, cartilage, adipocyte, fibroblast, marrow stroma, cardiac muscle, smooth muscle, skeletal muscle, pericyte, vascular, epithelial, glial, neuronal, astrocyte and oligodendrocyte cells.

In another embodiment, the MLPSCs are not capable of giving rise, upon culturing, to hematopoietic cells.

In one embodiment, MLPSCs are taken from a subject to be treated and cultured in vitro using standard techniques, e.g., prior to use in a treatment method as described herein according to any embodiment. Such cells are useful for administration to the subject in an autologous or allogeneic composition.

Culture-expanded MLPSCs may be obtained by culturing in any suitable medium known in the art, e.g., as described in WO 2004/85630. In an embodiment, progeny cells useful for the methods of the invention are obtained by isolating TNAP⁺ STRO-1⁺ multipotential cells from bone marrow using magnetic beads labelled with the STRO-3 antibody, and then culture expanding the isolated cells (see Gronthos et al. Blood 85: 929-940, 1995 for an example of suitable culturing conditions).

It will be understood that in performing the present invention, separation of cells carrying any given cell surface marker can be effected by a number of different methods, however, preferred methods rely upon binding a binding agent (e.g., an antibody or antigen binding fragment thereof) to the marker concerned followed by a separation of those that exhibit binding, being either high level binding, or low level binding or no binding. The most convenient binding agents are antibodies or antibody-based molecules, preferably being monoclonal antibodies or based on monoclonal antibodies because of the specificity of these latter agents. Antibodies can be used for both steps, however other agents might also be used, thus ligands for these markers may also be employed to enrich for cells carrying them, or lacking them.

The antibodies or ligands may be attached to a solid support to allow for a crude separation. The separation techniques preferably maximise the retention of viability of the fraction to be collected. Various techniques of different efficacy may be employed to obtain relatively crude separations. The particular technique employed will depend upon efficiency of separation, associated cytotoxicity, ease and speed of performance, and necessity for sophisticated equipment and/or technical skill. Procedures for separation may include, but are not limited to, magnetic separation, using antibody-coated magnetic beads, affinity chromatography and “panning” with antibody attached to a solid matrix. Techniques providing accurate separation include but are not limited to FACS. Methods for performing FACS will be apparent to the skilled artisan.

Antibodies against each of the markers described herein are commercially available (e.g., monoclonal antibodies against STRO-1 or TNAP) are commercially available from R&D Systems, USA), available from ATCC or other depositary organization and/or can be produced using art recognized techniques.

It is preferred that the method for isolating MLPSCs, for example, comprises a first step being a solid phase sorting step utilising for example magnetic activated cell sorting (MACS) recognising high level expression of STRO-1. A second sorting step can then follow, should that be desired, to result in a higher level of precursor cell expression as described in patent specification WO 01/14268. This second sorting step might involve the use of two or more markers.

The method obtaining MLPSCs cells might also include the harvesting of a source of the cells before the first enrichment step using known techniques. Thus the tissue will be surgically removed. Cells comprising the source tissue will then be separated into a so called single cells suspension. This separation may be achieved by physical and or enzymatic means.

In some embodiments MLPSCs including culture-expanded MLPSCs have been cryopreserved and thawed. In the most preferred embodiments MLPSCs used in the treatment methods disclosed herein are human MLPSCs.

### Culture Expansion of MLPSCs

In an example, MLPSCs are culture expanded. “Culture expanded” mesenchymal lineage precursor or stem cells media are distinguished from freshly isolated cells in that they have been cultured in cell culture medium and passaged (i.e. sub-cultured). In an example, culture expanded mesenchymal lineage precursor or stem cells are culture expanded for about 4-10 passages. In some embodiments the culture expanded MLPSCs are prelicensed by exposure to certain pro-inflammatory cytokines as disclosed herein. In an example, mesenchymal lineage precursor or stem cells are culture expanded for at least 5, at least 6, at least 7, at least 8, at least 9, at least 10 passages. For example, mesenchymal lineage precursor or stem cells can be culture expanded for at least 5 passages. In an example, mesenchymal lineage precursor or stem cells can be culture expanded for at least 5-10 passages. In an example, mesenchymal lineage precursor or stem cells can be culture expanded for at least 5-8 passages. In an example, mesenchymal lineage precursor or stem cells can be culture expanded for at least 5-7 passages. In an example, mesenchymal lineage precursor or stem cells can be culture expanded for more than 10 passages. In another example, mesenchymal lineage precursor or stem cells can be culture expanded for more than 7 passages. In these examples, stem cells may be culture expanded before being cryopreserved to provide an intermediate cryopreserved MLPSC population. In an example, compositions of the disclosure are prepared from an intermediate cryopreserved MLPSC population. For example, an intermediate cryopreserved MLPSC population can be further culture expanded prior to administration as is discussed further below. Accordingly, in an example, mesenchymal lineage precursor or stem cells are culture expanded and cryopreserved. In an embodiment of these examples, mesenchymal lineage precursor or stem cells can be obtained from a single donor, or multiple donors where the donor samples or mesenchymal lineage precursor or stem cells are subsequently pooled and then culture expanded. In an example, the culture expansion process comprises:
i. expanding by passage expansion the number of viable cells to provide a preparation of at least about 1 billion of the viable cells, wherein the passage expansion comprises establishing a primary culture of isolated mesenchymal lineage precursor or stem cells and then serially establishing a first non-primary (P1) culture of isolated mesenchymal lineage precursor or stem cells from the previous culture;
ii. expanding by passage expansion the P1 culture of isolated mesenchymal lineage precursor or stem cells to a second non-primary (P2) culture of mesenchymal lineage precursor or stem cells; and,
iii. preparing and cryopreserving an in-process intermediate mesenchymal lineage precursor or stem cells preparation obtained from the P2 culture of mesenchymal lineage precursor or stem cells; and,
iv. thawing the cryopreserved in-process intermediate mesenchymal lineage precursor or stem cells preparation and expanding by passage expansion the in-process intermediate mesenchymal lineage precursor or stem cells preparation.

In an example, culture expanded mesenchymal lineage precursor or stem cells are culture expanded for about 4-10 passages, wherein the mesenchymal lineage precursor or stem cells have been cryopreserved after at least 2 or 3 passages before being further culture expanded. In an example, mesenchymal lineage precursor or stem cells are culture expanded for at least 1, at least 2, at least 3, at least 4, at least 5 passages, cryopreserved and then further culture expanded for at least 1, at least 2, at least 3, at least 4, at least 5 passages before being administered or further cryopreserved.

In an example, mesenchymal lineage precursor or stem cells are culture expanded in a cell factory such as a 5 or 10 layer cell factory.

In an example, the majority of mesenchymal lineage precursor or stem cells in compositions of the disclosure are of about the same generation number (i.e., they are within about 1 or about 2 or about 3 or about 4 cell doublings of each other). In an example, the average number of cell doublings in the present compositions is about 20 to about 25 doublings. In an example, the average number of cell doublings in the present compositions is about 9 to about 13 (e.g., about 11 or about 11.2) doublings arising from the primary culture, plus about 1, about 2, about 3, or about 4 doublings per passage (for example, about 2.5 doublings per passage). Exemplary average cell doublings in present compositions are any of about 13.5, about 16, about 18.5, about 21, about 23.5, about 26, about 28.5, about 31, about 33.5, and about 36 when produced by about 1, about 2, about 3, about 4, about 5, about 6, about 7, about 8, about 9, and about 10 passages, respectively.

The process of mesenchymal lineage precursor or stem cell isolation and ex vivo expansion can be performed using any equipment and cell handing methods known in the art. Various culture expansion embodiments of the present disclosure employ steps that require manipulation of cells, for example, steps of seeding, feeding, dissociating an adherent culture, or washing. Any step of manipulating cells has the potential to insult the cells. Although mesenchymal lineage precursor or stem cells can generally withstand a certain amount of insult during preparation, cells are preferably manipulated by handling procedures and/or equipment that adequately performs the given step(s) while minimizing insult to the cells.

In an example, mesenchymal lineage precursor or stem cells are washed in an apparatus that includes a cell source bag, a wash solution bag, a recirculation wash bag, a spinning membrane filter having inlet and outlet ports, a filtrate bag, a mixing zone, an end product bag for the washed cells, and appropriate tubing, for example, as described in U.S. Pat. No. 6,251,295, which is hereby incorporated by reference.

In an example, a mesenchymal lineage precursor or stem cell composition according to the present disclosure is 95% homogeneous with respect to being CD105 positive and CD166 positive and being CD45 negative. In an example, this homogeneity persists through ex vivo expansion; i.e. though multiple population doublings. In an example, the composition comprises at least one therapeutic dose of mesenchymal lineage precursor or stem cells and the mesenchymal lineage precursor or stem cells comprise less than about 1.25% CD45+ cells, at least about 95% CD105+ cells, and at least about 95% CD166+ cells. In an example, this homogeneity persists after cryogenic storage and thawing, where the cells also generally have a viability of about 70% or more.

### Modified MLPSCs and Related Uses

In some embodiments the methods disclosed herein make use of modified (e.g., genetically modified MLPSCs). While not wishing to be bound by theory, it is known that MLPSCs (e.g., hMSCs) can delivery nucleic acids, e.g., miRNAs, antagomiRs, siRNAs, to other cells, e.g., target cells via both gap junctions, exosomes, and extracellular vesicles. In some embodiments the modified MLPSCs comprise at least one of an exogenous anti-inflammatory miRNA, antagomiR, siRNA, RNAi, antisense oligonucleotide, or antisense RNA, which can be useful in further facilitating partial reprogramming of target cells. In some embodiments the modified MLPSCs comprise one or more exogenous anti-inflammatory miRNAs selected from the group consisting of: miR-10a, miR-21, miR-24, miR-124, miR-145, miR-146, miR-149, and miR-181a-miR-181d. In other embodiments the modified MLPSCs comprise one or more exogenous anti-inflammatory antagomiRs selected from the group consisting of: a miR-146b-5p, a miR-34a antagomir, a miR-21 antagomir, a miR-126 antagomir, a miR-221 antagomir, a miR-338-3p antagomir, and a miR-155 antagomir. In some embodiments the modified MLPSCs comprise one or more exogenous anti-inflammatory siRNAs, antisense oligonucleotides, or antisense RNAs targeting one or more targets selected from the group consisting of: TNF-α, IL-1β, and IL-18.

In some embodiments the modified MLPSCs comprise at least one of an exogenous siRNA, RNAi, antisense oligonucleotide, or antisense RNA directed against at least one of: p16^{INK4a}, p21^{WAF1/CIP1}, the mTOR signalling pathway, or the c-Jun N-terminal kinase (JNK) signalling pathway. In some preferred embodiments, the modified MLPSCs comprise an exogenous siRNA or antisense oligonucleotide against one or more of p16^{INK4a}, p21^{WAF1/CIP1}, or mTOR.

In some embodiments the modified MLPScs comprising an miRNA selected from the group consisting of: mir-302b, mir-302c, mir-302a, mir-302d, and mir-367, which can increase the efficiency of partial reprogramming.

In some embodiments the modified MLPSCs are genetically modified MLPSCs comprising one or more exogenous nucleic acids encoding one or more reprogramming factors or one or more targeted transactivators, wherein:
(i) the modified MLPSCs, when in the presence of one or more target cells, deliver the one or more exogenous nucleic acids to the one more target cells;
(ii) the one or more encoded reprogramming factors are expressed in the one or more target cells at a level sufficient to partially reprogram the one or more target cells; and
(iii) the one or more encoded reprogramming factors or targeted transactivators are substantially inoperable to expression in the modified MLPSCs prior to the delivery in (i), so as to avoid reprogramming of the modified MLPSCs themselves. While not wishing to be bound by theory, it is generally understood in the art that mRNAs can be delivered intercellularly via tunnelling nanotubes. See, e.g., Haimovich et al. (2021), Biochem Soc Trans., 49(1):145-160. Thus, in some preferred embodiments the genetically modified MLPSCs are loaded with one or more synthetic mRNAs encoding one or more reprogramming factors or targeted transactivators as described herein, wherein the encoded reprogramming factors are fusion proteins comprising a CDD as described herein, which, in the absence of the stabilization ligand (e.g., trimethoprim), prevents accumulation of the encoded proteins. Such a configuration permits delivery of the synthetic mRNAs to target cells.

Efficiencies of genetic modification are rarely 100%, and it is usually desirable to enrich the population for cells that have been successfully modified. In an example, modified cells can be enriched by taking advantage of a functional feature of the new genotype. One exemplary method of enriching modified cells is positive selection using a selectable or screenable marker gene. “Marker gene” refers to a gene that imparts a distinct phenotype to cells expressing the marker gene and thus, allows such transformed cells to be distinguished from cells that do not have the marker. A selectable marker gene confers a trait for which one can “select” based on resistance to a selective agent (e.g., an antibiotic such as puromycin or blasticidin). A screenable marker gene (or reporter gene) confers a trait that one can identify through observation or testing, that is, by “screening” (e.g., β-glucuronidase, luciferase, GFP or other enzyme activity not present in untransformed cells). In an example, genetically modified mesenchymal lineage precursor or stem cells are selected based on resistance to a drug such as neomycin or colorimetric selection based on expression of lacZ.

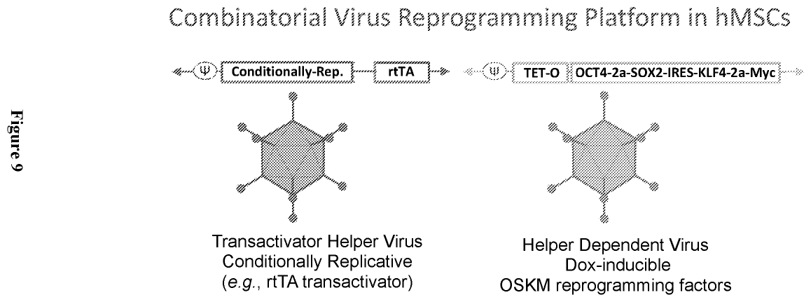

In some embodiments the above-mentioned genetically modified MLPSCs are provided as a composition for enhancing partial reprogramming of target cells, which also serves as a delivery platform (e.g., a combinatorial helper/helper-dependent virus platform; FIG. 9) for delivering to target cells in a subject exogenous nucleic acids encoding one or more: (a) reprogramming factors; or (b) targeted transactivators that induce expression of one or more endogenous reprogramming factors. As described above, by judicious selection of the encoding nucleic acids, transfer of the nucleic acids from the genetically modified MLPSCs occurs prior to expressing reprogramming factors at functionally relevant levels in the target cells. This allows the MLPSC-5 specific enhancement of partial reprogramming to occur with minimal or reduced risk of MLPSCs themselves acquiring an altered phenotype or cell identity by premature reprogramming factor expression. In some embodiments the composition for enhanced partial reprogramming is provided as a pharmaceutical composition comprising the genetically modified MLPSCs and a pharmaceutically acceptable excipient.

Exosomes Derived from MLPSCs

In some embodiments the treatment methods disclosed herein include administering purified exosomes or extracellular vesicles isolated from MLPSCs or modified MLPSCs.

In other embodiments provided herein are compositions containing exosomes derived from a high efficacy hMSC population. In some embodiments such extracellular vesicles or exosomes are generated by preparing from any of the high efficacy hMSC populations described herein.

Methods for obtaining clinical grade extracellular vesicles and exosomes are known in the art. See, e.g., Park et al., (2019), Stem Cell Research & Therapy, 10:288; and Yin et al., (2019), Biomarker Research, 7:8.

For example, exosomes can be isolated from MLPSCs grown to about 70% confluence in exosome-depleted serum-containing medium over a period of about 24-48 hours. The resulting culture medium is then centrifuged 2-3 times at low speed (500×g-800×g) for 15 minutes-30 minutes. Afterwards, the resulting supernatant is spun 2-3 times at higher speed (about 2000×g to 4000×g) for about 15 minutes-30 minutes, and the supernatant from this spin is collected and spun a third time at about 10,000×g to 15,000×g for approximately 30 minutes. This supernatant is collected and transferred to, e.g., Ultra-Clear centrifuge tubes (Beckman Coulter) and centrifuged at 70,000×g for 1-2 hours at 4° C. in a SW32Ti rotor (Beckman Coulter) or equivalent rotor. The supernatant is carefully decanted, the exosomal fraction pellet is resuspended in a physiological saline buffer (e.g., PBS) and the ultra-centrifugation cycle is repeated. Afterwards, the exosomal pellet is gently resuspended in about 100 μl to about 500 μl of physiological buffer. The resuspended exosomes can be stored at −80° C. if necessary.

In an exemplary embodiment for isolation of an extracellular vesicle fraction, MLPSC supernatant obtained as described in the above example is collected and centrifuged at 400×g-600×g for 10 minutes, and subsequently at 2000×g for 20 minutes. At each of these steps, the supernatant is collected and transferred to new 5 tubes for the next step and the resulting pellet is discarded. The final supernatant from these low speed spins is ultracentrifuged in serial spins of increasing speed as follows: 10,000×g, 20,000×g, 40,000×g and 60,000×g in a 12110 angle rotor (Sigma, 3K30, Germany) or equivalent rotor for about 60 minutes to 90 minutes at 4° C. The pellet is then washed about 3 to 4 times using a physiological buffer, and the ultracentrifugation steps are repeated. Afterwards, the resulting pellet can be resuspended in a physiological buffer and be used or stored at −80° C.

### Administration of MLPSCs, Exosomes, or Conditioned Media

The MLPSCs, e.g., STRO-1l cells, MSCs, culture-expanded multipotential progeny thereof, exosomes derived therefrom, or MLPSC-conditioned media can be administered by any of a number of routes with due consideration of the location, distribution, and circulatory access of the population of target cells to be partially reprogrammed and the target tissue(s) in which they reside.

In some embodiments, the MLPSCs are administered to the blood stream of a subject, e.g., parenterally. Exemplary routes of parenteral administration include, but are not limited to, intra-arterial, intravenous, or intraperitoneal routes of administration. In some embodiments systemic administration of MLPSCs is where target cells to be partially reprogrammed are in the liver or in a kidney.

In some embodiments, where the population of target cells is in the heart, administration is carried out intra-arterially, into an aorta, into an atrium, or into a ventricle of the heart. In the case of cell delivery to an atrium or ventricle of the heart, cells can be administered to the left atrium or ventricle to avoid complications that may arise from rapid delivery of cells to the lungs. In one embodiment, the population is administered into the carotid artery. In another embodiment, the population is administered into the myocardium. In an example, the population is administered into inflamed myocardium. In certain examples, inflamed myocardium is identified using a suitable mapping catheter before cells are administered to one or more of the identified sites of inflammation.

In other embodiments, where the target cells are present within an intervertebral disc, MLPSCs are administered within the intradiscal space or within the nucleus pulposus. In other embodiments, where the target cells are present within a joint, the MLPSCs are administered by intra-articular injection. In other embodiments, where the target cells are located within the brain, MLPSCs are administered by an intracerebral route, an intraventricular route, an intrathecal route, or by an intra-carotid route of administration.

In other embodiments administration is intramuscular. In still other embodiments administration of MLPSCs is intradermal.

In some embodiments, administration of MLPSCs and or exosomes derived therefrom is done in a single bolus dose. In other embodiments administration is by continuous infusion, or by doses at intervals of, e.g., one day, one week, or 1 to 7 times per week. An exemplary dose protocol is one involving the maximal dose or dose frequency that avoids significant undesirable side effects. A total weekly dose depends on the type and activity of the factors/cells being used. Determination of the appropriate dose is made by a clinician, e.g., using parameters or factors known or suspected in the art to affect treatment or predicted to affect treatment and/or success of target cell partial reprogramming. Generally, the dose begins with an amount somewhat less than the optimum dose and is increased by small increments thereafter until the desired or optimum effect is achieved relative to any negative side effects.

In some embodiments MLPSCs or exosomes derived from the MLPSCs are administered prior to initial expression of the one or more reprogramming factors to be expressed in a subject in the methods disclosed herein. In some embodiments the initial administration is provided between about 21 days to about one hour prior to initial expression of the one or more reprogramming factors in the subject, e.g., 20 days, 18 days, 16 days, 14 days, 12 days, 10 days, 8 days, 7 days, 5 days, 3 days, 2 days, 1 day, 12 hours, 8 hours, 3 hours, or another time point prior to initial expression of the one or more reprogramming factors from about 21 days to about one hour prior to the initial expression of the one or more reprogramming factors. In some embodiments no further administration of MLPSCs is provided following initiation of expression of the one or more reprogramming factors. In some embodiments MLPSCs are administered during at least one period of expression of the one or more reprogramming factors. In some embodiments, MLPSCs are administered after initiation of expression of the one or more reprogramming factors over a period of about 1 day to about 21 days during expression of the one or more reprogramming factors, e.g., 2 days, 3 days, 4 days, 7 days, 10 days, 12 days, 14 days, 18 days, 20 days, or another period from about 1 day to about 21 days during expression of the one or more reprogramming factors. In other embodiments MLPSCs or exosomes derived therefrom are administered after a period of expression of the one or more reprogramming factors has ended and expression has subsided, e.g., within a period from about one day to about two weeks after reprogramming factor expression has subsided in the population of target cells, e.g., 2 days, 3 days, 5 days, 7 days, 10 days, 12 days, or another period from about one day to about two weeks after reprogramming factor expression has subsided in the population of target cells.

A suitable dose of MLPSCs for the partial reprogramming methods disclosed herein will depend on a number of factors including, but not limited to, the route of administration, the target cell population to be partially reprogrammed, target cell tissue density, the age, gender, weight, and severity of a health condition of the subject undergoing the treatment.

In some embodiments, where MLPSCs are to be administered systemically, about 1×10⁵ MLPSCs/kg to about 1×10⁸ MLPSCs/kg of subject weight are administered. In some embodiments 1×10⁶ MLPSCs/kg to about 5×10⁶ MLPSCs/kg are administered. In some embodiments 1×10⁷ MLPSCs/kg to about 5×10⁷ MLPSC/kg are administered.

In other embodiments a suitable dose of MLPSCs to be administered is based on an assessed volume of tissue comprising a population of target cells to be partially reprogrammed in a volume of tissue affected by a health condition (e.g., heart failure, stroke, kidney failure, or IVD), e.g., about 2×10⁶ MLPSCs/cm³ of affected tissue to about 2×10⁷ MLPSCs/cm³ of affected tissue, e.g., 3×10⁶, 4×10⁶, 5×10⁶, 8×10⁶, 1.2×10⁷, 1.5×10⁷, or another number of cells/cm³ from about 2×10⁶ MLPSCs/cm³ to about 2×10⁷ MLPSCs/cm³ of affected tissue.

In other embodiments dosing is based on number of MLPSC to be administered that corresponds to a relative percentage of the number of cells in the target population to be partially reprogrammed, e.g., about 0.5% to about 50% of the number of target cells to be partially reprogrammed, e.g., 1%, 2%, 5%, 7%, 10%, 15%, 20%, 30%, 40%, 45%, or another percent of the number of target cells to be partially reprogrammed. For example in a human subject suffering from congestive heart failure and in need of partial reprogramming of cardiomyocytes, the number of administered MLPSCs, e.g., intracardiac administration of MLPSCs could be about 1% of total cardiomyocytes in a human heart (about 2-3 billion cardiomyocytes), i.e., about 2×10⁷ to 3×10⁷ MLPSCs.

In some embodiments instead of, or in addition to MLPSCs, a subject is administered isolated exosomes or extracellular vesicles derived from MLPSCs (e.g., hMLPSCs). In some embodiments extracellular vesicles or exosomes derived from a In some embodiments, an effective amount of extracellular vesicles or exosomes for partial reprogramming is about 50 μg (extracellular vesicle protein or exosome protein content) to about 1,000 μg (extracellular vesicle protein or exosome protein content), e.g., 60 μg, 70 μg, 80 μg, 100 μg, 250 μg, 300 μg, 400 μg, 500 μg, 600 μg, 800 μg, or another amount of extracellular vesicles or exosomes from about 50 μg (extracellular vesicle protein or exosome protein content) to about 1,000 μg (extracellular vesicle protein or exosome protein content).

In some embodiments, MLPSCs or exosomes/extracellular vesicles derived therefrom are administered in a composition comprising a carrier or excipient.

Suitable carriers for the present disclosure include those conventionally used, e.g., saline, aqueous dextrose, lactose, Ringer's solution, a buffered solution, hyaluronan and glycols are exemplary liquid carriers, particularly (when isotonic) for solutions. In some embodiments, the administered composition includes a pharmaceutically acceptable amount of a residual cryopreservative agent.

In another example, a carrier is a media composition, e.g., in which a cell is grown or suspended. In an example, such a media composition does not induce any adverse effects in a subject to whom it is administered.

Exemplary carriers and excipients do not adversely affect the viability of a cell.

In one example, the carrier or excipient provides a buffering activity to maintain the cells at a suitable pH to thereby exert a biological activity, e.g., the carrier or excipient is phosphate buffered saline (PBS). PBS represents an attractive carrier or excipient because it interacts with cells and factors minimally and permits rapid release of the cells and factors, in such a case, the composition of the disclosure may be produced as a liquid for direct application to the blood stream or into a tissue or a region surrounding or adjacent to a tissue, e.g., by injection.

In some embodiments different subpopulations of MLPSCs are administered in the partial reprogramming methods disclosed herein. In some, embodiments a mixture of a modified MPLSCs, e.g., containing exogenous miRNAs, and a non-modified population of MLPSCs are administered to a subject in the methods disclosed herein.

In some embodiments different populations of MLPSCs are administered to a subject at different time points. For example, MLPSCs comprising an siRNA against p16^{INK4a} may be provided just before the beginning of expression of reprogramming factors in a population of target cells in the subject, and a population of unmodified MLPSCs administered after a few days of reprogramming factor expression.

As will be appreciated, the enhanced partial reprogramming methods described herein are useful for tissue reconstitution or regeneration in a human patient in need thereof. For example, the efficacy of enhance partial reprogramming in spinal cord injury neural cell transplants can be assessed in a rat model for acutely injured spinal cord, as described by McDonald, et al. ((1999) Nat. Med., vol. 5:1410) and Kim, et al. ((2002) Nature, vol. 418:50). Successful partial reprogramming will show new astrocytes, oligodendrocytes, and/or neurons, migrating along the spinal cord from the lesioned end, and an improvement in gait, coordination, and weight-bearing.

Similarly, the efficacy of enhanced partial reprogramming can be assessed in a suitable animal model of cardiac injury or dysfunction, e.g., an animal model for cardiac cryoinjury where about 55% of the left ventricular wall tissue becomes scar tissue without treatment (Li, et al. (1996), Ann. Thorac. Surg., vol. 62:654; Sakai, et al. (1999), Ann. Thorac. Surg., vol. 8:2074; Sakai, et al. (1999), J. Thorac. Cardiovasc. Surg., vol. 118:715). Successful treatment will reduce the area of the scar, limit scar expansion, and improve heart function as determined by systolic, diastolic, and developed pressure (Kehat, et al. (2004)). Cardiac injury can also be modeled, for example, using an embolization coil in the distal portion of the left anterior descending artery (Watanabe, et al. (1998), Cell Transplant., vol. 7:239), or by ligation of the left anterior descending coronary artery (Min, et al. (2002), J. Appl. Physiol., vol. 92:288). Efficacy of treatment can be evaluated by histology and cardiac function. Cardiomyocyte preparations embodied in this invention can be used in therapy to regenerate cardiac muscle and treat insufficient cardiac function.

Liver function can also be restored by enhanced partial reprogramming by the methods disclosed herein. The outcome of enhanced partial reprogramming in damaged liver can be assessed in animal models for ability to repair liver damage.

The present invention is described further in the following non-limiting examples.

### EXAMPLES

### Example 1: Effect of MLPSCs on Partial Reprogramming Efficiency of Fibroblasts Using Lentiviruses

### MLPSC Populations

As an exemplary MLPSC population, STRO-1⁺ multipotential progenitor cells (MPCs) are isolated from bone marrow by immunoselection using the STRO3 mAb, and then culture-expanded and cryopreserved in ProFreeze™-CDM (Lonza, USA), essentially as described in Gronthos and Zannettino Methods Mol Biol. 449:45-57, 2008). For evaluating the effect of MLPSCs on partial reprogramming efficiency, fibroblasts previously transduced with the reprogramming factors OCT4, KLF4, SOX2, and c-MYC (“OKSM”) are co-cultured with allogeneic STRO-1+ MPCs at varying ratios of MPC to fibroblasts. Passage 4 MPCs are thawed and constituted in vehicle for immediate use.

### Fibroblasts

Primary human skin fibroblast lines—GM09503 (passage frozen (P), P3), GM01651 (P14), GM01681 (P12), and GM01680 (P12) are purchased from the Coriell Institute (Camden, NJ, USA). The fibroblasts are cultured in MEM (Gibco) supplemented with 15% (v/v) heat inactivated FBS (Gibco), 1% (v/v) MEM non-essential amino acids (Gibco), and 1% (v/v) Pen-Strep (Gibco) in a humidified cell culture incubator (37° C., 5% CO₂).

### Effect of MPCs on Partial Reprogramming Efficiency of Human Dermal Fibroblasts

Cultures of human dermal fibroblasts plated on rh-Laminin-521 (ThermoFisher) are transduced with two lentiviruses: (1) a doxycycline-inducible polycistronic OKSM reprogramming vector; and (2) an EF-2α constitutive rtTA expression vector. The lentiviruses are added to the fibroblast cultures at a multiplicity of infection (MOI) of about 5 after addition of polybrene carrier at a final concentration of about 6 μg/ml (Sigma).

The lentivirus-containing medium is replaced with fresh fibroblast medium (10% FBS) containing doxycycline (2 μg/ml) the next day, and MPCs are added to a subset of the transduced fibroblast cultures at MPC to fibroblast ratios of 0.1, 0.5, 1, and 3. After co-culture for seven days in the presence of doxycycline, the cultures are analysed by flow cytometry for expression of the early reprogramming marker TRA-1-60. Co-cultures of MPCs and untransduced fibroblasts are used as a negative control. The proportion of TRA-1-60⁺ cells in each condition is used as an indicator of relative reprogramming efficiency. It is expected that the presence of MPCs will increase reprogramming efficiency as reflected by the proportion of TRA-1-60⁺ cells.

### Example 2: Effect of Mlpscs on mRNA-Based Partial Reprogramming Efficiency

Reprogramming of somatic cells using mRNA-based reprogramming is generally considered to have essentially no associated risk of genomic integration/mutational events. Thus, mRNA-based partial reprogramming is likely to be a safe approach for in vivo/therapeutic applications. Accordingly, we evaluate the effect of MLPCs on mRNA-based partial reprogramming of aged human dermal fibroblasts in vitro.

### Fibroblasts

Human adult dermal fibroblasts as described above are cultured in Eagle's Minimum Essential Medium with Earl's salts supplemented with nonessential amino acids, 10% FBS, and 1% Penicillin/Streptomycin. Cells are cultured at 37° C. with 5% CO₂.

mRNA Transfection and MLPSC Co-Culture

Fibroblasts from individuals aged 60-90 years are cultured on iMatrix-511 and transfected using Lipofectamine MessengerMAX™ using the manufacturer's protocol. During the transfection period, NutriStem® serum-free medium (Sartorious 05-100-1A) is used. A cocktail of mRNAs encoding the reprogramming factors OCT4, SOX2, KL4, c-MYC, NANOG, and LIN-28 provided in the StemRNA™ 3^{rd} Generation Reprogramming Kit (Reprocell 00-0076). Following the fourth day of transfection, the culture medium is replaced with 1:1 mixture of StemFit for MSC medium (Reprocell AS-MSC) and NutriStem medium containing MPCs as described in Example 1 for a final ratio of MPCs to fibroblasts of 0.1, 0.5, 1, and 3. The co-cultures are then maintained and passaged as needed for a total of ten days from the initial fibroblast transfection. Afterwards, the cultures are analysed by flow cytometry for expression of the early reprogramming marker TRA-1-60. Co-cultures of MPCs and untransduced fibroblasts are used as a negative control. The proportion of TRA-1-60+ cells in each condition is used as an indicator of relative reprogramming efficiency. It is expected that the presence of MPCs will increase reprogramming efficiency as reflected by the proportion of TRA-1-60⁺ cells.

### Determination of Epigenetic Age Changes During Partial Reprogramming

Epigenetic clocks based on DNA methylation levels are useful molecular biomarkers of age across tissues and cell types and are predictive of a host of age-related conditions including lifespan (Horvath et al. 2013, Genome Biol., 14:R115). Exogenous expression of canonical reprogramming factors (OSKM) is known to revert the epigenetic age of primary cells to a prenatal state (Horvath et al., supra). To determine the effect of MLPSC co-culture modulated partial reprogramming, two epigenetic clocks that apply to human fibroblasts are used: Horvath's original pantissue epigenetic clock (based on 353 cytosine-phosphate-guanine pairs), and the more recent skin-and-blood clock (based on 391CpGs)-Horvath et al., supra.

Genomic DNA samples isolated from fibroblast cultures prior to transfection, and TRA-1-60+ selection at 6 days, 8 days, 12 days, and 15 days following initiation of transfection are analysed for methylation patterns on a human Illumina Infinium EPIC 850K chip. A previously defined mathematical algorithm is used to combine the methylation levels of 353 CpG into an age estimate (in units of years), which is referred to as epigenetic age or “DNAm age” (Horvath et al., supra). A secondary analysis is also performed using on the skin-and-blood epigenetic clock (based on 391 CpGs) because it is known to lead to more accurate DNAm age estimates in fibroblasts and several other cell types. An online version of the epigenetic clock software is used to arrive at DNA methylation age estimates.

### Example 3: Effect of MLPSCs on Adenovirus-Based Based Partial Reprogramming of Cardiomyocytes In Vivo

### Inducible Adenovirus for Expression of Reprogramming Factors In Vivo

In order to perform non-integrative partial reprogramming in vivo, we construct a regulatable adenovirus for simultaneous expression of the reprogramming factors OCT4, SOX2, KLF4, and c-MYC (“OKSM”) genes and a red fluorescent protein (mCherry) based on the construct described in Lehmann et al., (2019), Gene Therapy, 26:432-440.

The adenoviral vector was is constructed using a commercial kit (Microbix Inc., Ontario, Canada) that provides the shuttle plasmid pC4HSU, the helper adenovirus H14 and the HEK293 Cre4 cell line. As starting point we use a regulatable Tet-On bidirectional construct (pTRE3G-BI-mCherry; Takara Cat No. 631333) the gene for mCherry in one of two multiple cloning sites (MCS) flanking the bidirectional promoter PₘᵢₙCMV-TRE-PₘᵢₙCMV. On a separate site on the construct, a constitutive expression cassette (CMV-rtTA) that expresses the DOX-activated (“reverse tetracycline transactivator”) rtTA is cloned. In the second MCS, we clone in the bicistronic tandem OCT4-f2A-KLF4-IRES-SOX2-p2A-Cmyc. The encoded reprogramming factors are grouped in pairs placed downstream and upstream of an internal ribosome entry site (IRES), and each pair of reprogramming factors ORFs is separated by a type 2A CHYSEL (cis-acting hydrolase element) self-processing short sequence that causes the ribosome to skip the Gly-Pro bond at the C-terminal end of the 2A sequence, thus releasing the peptide upstream the 2A element but continuing with the translation of the downstream mRNA sequence. This allows near stoichiometric co-expression of the two cistrons flanking a 2A-type sequence. For reference, this construct is called prtTA-TRE-OKSM-mCherry.

Next, the whole bidirectional TRE-OKSM-mCherry expression cassette is excised and cloned into the pC4HSU shuttle vector to generate pC4HSU-rtTA-TRE-OKSM-mCherry. The pC4HSU shuttle consists of the inverted terminal repeats (ITRs) for Ad 5 virus, the packaging signal and part of the E4 adenoviral region plus a stuffer noncoding DNA of human origin which keeps a suitable size (28-31 Kbp) of the viral DNA. Between the two ITRs there is a bacterial sequence flanked by Pme I sites. The pC4HSU-rtTA-TRE-OKSM-mCherry plasmid is digested with Pme I in order to remove the bacterial sequence, thus generating the desired HD-RAd-rtTA-TRE-OKSM-mCherry genome (“Ad-reprogram”).

The linearized DNA backbone of the Ad-reprogram construct is transfected into Cre 293 cells. Next day, purified helper H14 virus was added to the cell cultures at a multiplicity at an MOI of 5. In H14, the packaging signal is flanked by lox P sites recognized by the Cre recombinase expressed by the 293 Cre4 cells. Therefore, the helper virus provides in trans all of the viral products necessary for generation of the desired Ad-reprogram virus. The infected 293 Cre4 cells are left for 2-3 days until cytopathic effect (CPE) is evident. Cells and medium are collected and subjected to three freeze-thaw cycles to lyse them. Clear lysates are obtained, mixed with H14 helper virus and added to a fresh culture of Cre4 293 cells at a MOI of 1. When CPE appears, passage 2 (P2) cell lysates are prepared. This iterative coinfection process is repeated five more times in order to generate a sufficient number of Adenoviral particles for the purification step. The newly generated adenoviral particles are rescued from P6. The Ad-reprogram viral particles so generated are purified by ultracentrifugation on CsCl gradients. Final virus stock is titrated by lysing the viral particles, extracting their DNA and determining its concentration in a Nanodrop spectrophotometer. Titer following purification is approximately 1×10¹² viral particles/ml. A control adenovirus with a Dox-inducible EGFP (Ad-EGFP) was used as a negative control as described below.

Virus stocks are resuspended in phosphate buffered saline (PBS) containing 5% glycerol and were diluted in in physiological saline prior to injection.

### Intramyocardial Injection of Ad-Reprogram and MLPSCs

Groups 1-5 of thymic nude rats (n=6 each) undergo left anterior descending coronary artery (LAD) ligation to induce a myocardial infarction. Two days later (“day 0” groups of animals (1-5) as indicated in Table 1 below are sham injected (groups 1, 2), injected with 1 μl (1×10⁹ viral particles) of Ad-reprogram (groups 3, 4) or Ad-EGFP (groups 5,6). The following day (“day 1”), groups of animals as set out below are provided with drinking water either containing 0.2 mg/ml of Doxycycline hyclate supplemented with 7.5% sucrose (groups 3-6); or with 7.5% sucrose alone (groups 1, 2) for a period of 7 days. The day after induction of reprogramming factor expression (“day 2”), each of the indicated groups is administered 1×10⁶ MLPSCs by direct intramyocardial injection (2, 4, 6) or sham-injected (1, 3). On days 10-15 global systolic and diastolic parameters of cardiac function are determined. On day 16, animals are sacrificed and neovascularization and immunostaining and cytometry are performed for cardiomyocytes, and co-localised mitotic markers.

TABLE 1
Experimental Design for MLPSC-Enhanced Partial Reprogramming
Therapy in an Ischemic Heart Model

| Group | Virus | Dox | MLPSCs | n |
|---|---|---|---|---|
| 1 | − | − | − | 6 |
| | (sham | | (sham | |
| | injection) | | injection) | |
| 2 | − | − | + | 6 |
| | (sham | | | |
| | injection) | | | |
| 3 | Ad-Reprogram | + | − | 6 |
| | | (7 days) | (sham | |
| | | | injection) | |
| 4 | Ad-Reprogram | + | + | 6 |
| | | (7 days) | | |
| 5 | Ad-EGFP | + | − | 6 |
| | (Dox- | 7 days | (sham | |
| | inducible) | | injection) | |
| 6 | Ad-EGFP | + | + | 6 |
| | (Dox- | 7 days | | |
| | inducible) | | | |

### Example 4: Serum Analysis

Mesenchymal precursor lineage or stem cell populations were culture expanded in either 5% FCS/5% NBCS (serum A) or 10% fetal bovine serum (serum B). These MLPSCs were used in example 7.

Cytokine levels in 5% FCS/5% NBCS (serum A) and 10% fetal bovine serum (serum B) were assessed. To provide an external control, cytokine levels were also assessed in FBS from a different supplier (serum C). In each instance, cytokine levels were assessed in neat serum.

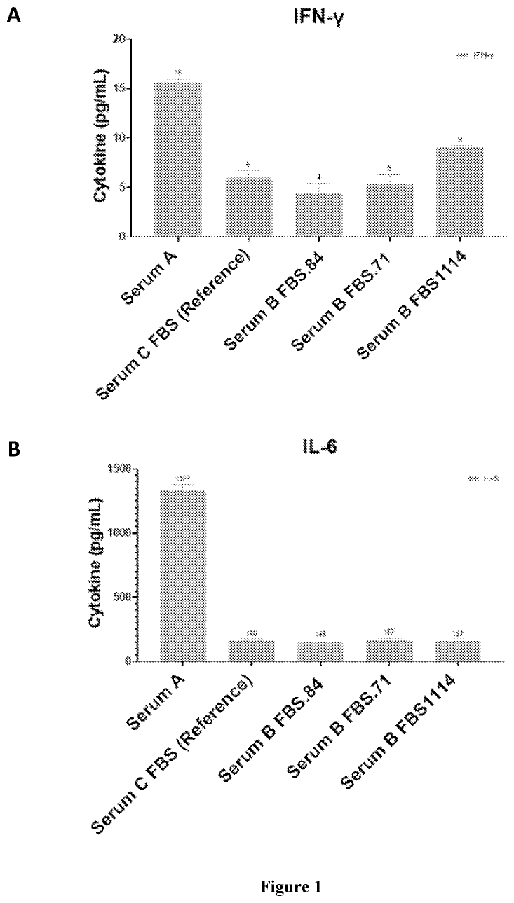

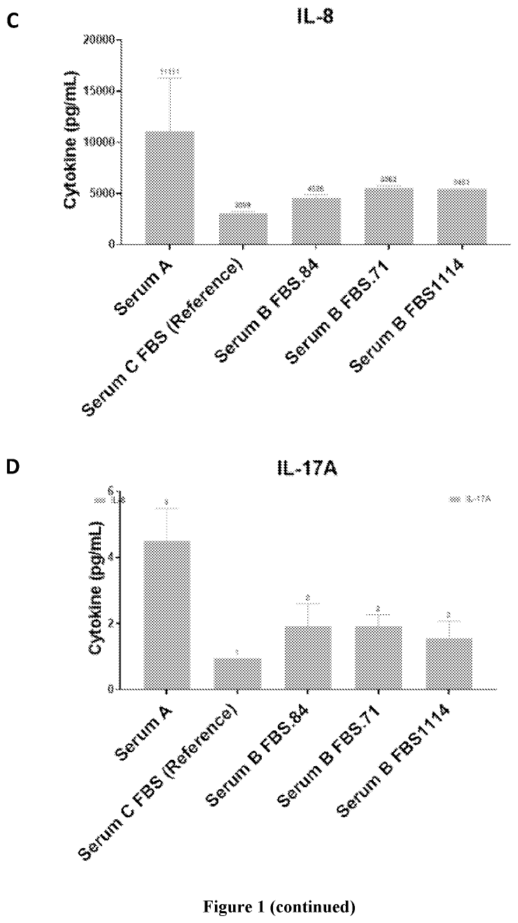

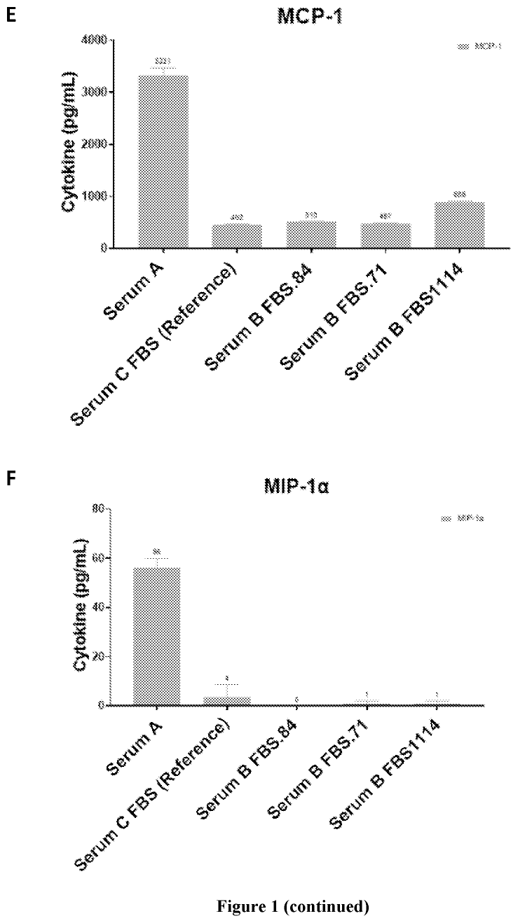

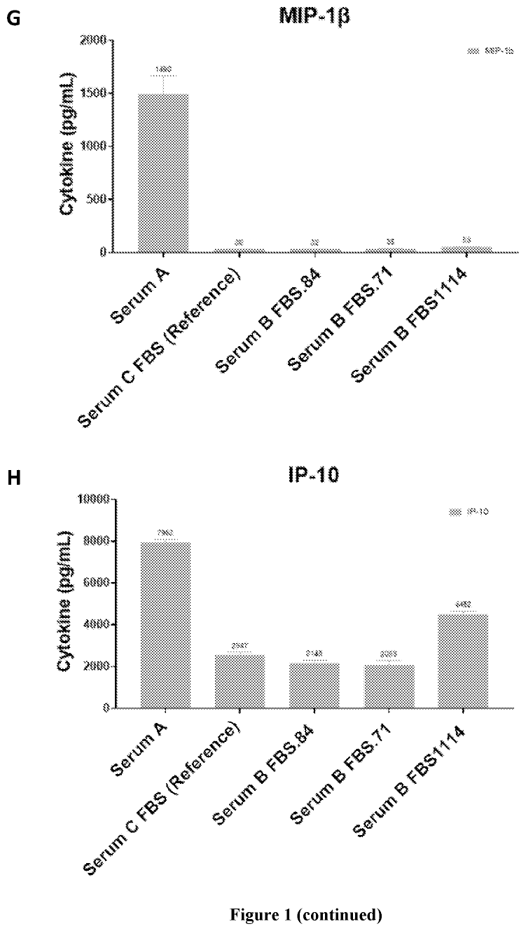

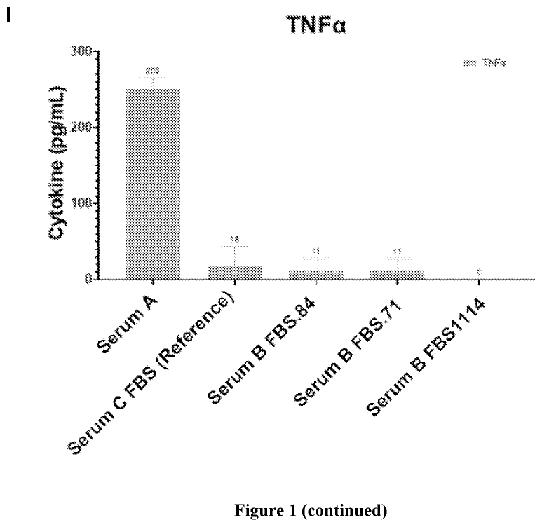

Surprisingly, pro-inflammatory cytokine levels were higher in serum preparations containing newborn calf serum (FIG. 1). Notably, there was an increase in pro-inflammatory cytokines known to bind receptors expressed on the surface of MLPSCs, such as interferon gamma (IFNγ), tumor necrosis factor alpha (TNFα) and, interleukins. For example, the following was observed in serum preparations containing newborn calf serum relative to fetal bovine serums:
At least a 2× increase in IFNγ;
At least an 13× increase in TNFα;
At least an 8× increase in IL-6;
At least an 2× increase in IL-8;
At least an 2× increase in IL-17A.

### Example 5: MLPSC Compositions Derived Using Culture Media Comprising Fetal Serum

The Alpha modification of Eagle's minimum essential media (MEM) with Earle's balanced salts, commonly referred to as Eagle's Alpha MEM, contains non-essential amino acids, sodium pyruvate, and additional vitamins. These modifications were first described for use in growing hybrid mouse and hamster cells (Stanners et al. 1971).

Eagle's Alpha MEM media suitable for culturing primary stem cells can be obtained from a variety of sources, including Life Technologies and Sigma.

A detailed method of establishing primary stem cell cultures, including the required growth factors used in the Exemplified processes is described in Gronthos and Simmons 1995.

Eagle's Alpha MEM media supplemented with 10% fetal calf serum, L ascorbate-2-phosphate (100 μM), dexamethasone (10⁷ M) and/or inorganic phosphate (3 mM) was used for culturing MLPSCs.

### Example 6: Pre-Licensing Culture Media (Newborn Serum Culture Media)

For the MLPSC culture media comprising newborn serum, the serum component of the Eagle's Alpha MEM culture media described in Example 2 was modified by supplementing with 5% (v/v) newborn serum (Differences in the fetal serum media and newborn serum media are shown in Table 2). The newborn serum used was newborn calf serum (NBCS; serum A). NBCS was 100% o bovine serum obtained from animals meeting the standard fetal bovine serum specifications but under the age of 20 days after birth.

NBCS was obtained from a commercial supplier, where it is marketed as an FCS substitute that is highly similar to FCS, to be used interchangeably, and expected to perform the same on cell lines.

TABLE 2
Summary of the differences between fetal serum
culture media and licensing culture media

| | |
|---|---|
| Fetal serum | |
| culture media | Licensing culture media |

Media (Change applicable to Thaw Feed, Passage)

| | |
|---|---|
| Alpha MEM | 50 mg/L short acting ascorbic acid derivative (Sodium |
| 10% v/v FCS | L-ascorbate) replaces long acting ascorbic acid |
| | derivative (L-ascorbic acid-2-phosphate) |
| | 5% v/v FCS and 5% v/v newborn serum |

Cryopreservation Formulation (50% Alpha-MEM/42.5% ProFreeze/
7.5% DMSO)

| | |
|---|---|
| Alpha MEM | 50 mg/L short acting ascorbic acid derivative (Sodium |
| 10% v/v FCS | L-ascorbate) replaces long acting ascorbic acid |
| | derivative (L-ascorbic acid-2-phosphate) |
| | 5% v/v FCS |
| | 5% v/v newborn calf serum |

### Example 7: Assessment of MPCs Cultured in Newborn Serum-Supplemented Medium Versus Fetal Serum Only Medium: Treatment Efficacy in Context of Persistent Inflammation

NYHA Class II/III high-risk heart failure with reduced ejection fraction (HFrEF) is a clinical model of persistent inflammation. HFrEF patients are characterized by cardiac and systemic inflammation, as determined by the presence of elevated inflammatory biomarkers. MPCs cultured under different serum conditions were administered to HFrEF patients in the clinical study described below.

In HFrEF patients, cardiac macrophages produce high levels of pro-inflammatory cytokines (IL-6, IL-1, TNF-alpha) which cause endothelial dysfunction and cardiomyocyte apoptosis. Plasma C-Reactive Protein (CRP) levels, as determined by a high sensitivity CRP (hsCRP) assay, reflect hepatic production of acute phase reactants in response to the high levels of pro-inflammatory cytokines (IL-6, IL-1 and TNF-alpha) produced by cardiac macrophages. Accordingly, plasma hsCRP levels (<2 mg/L vs >2 mg/L) are representative systemic measurements reflective of low or high intra-cardiac inflammation. In the following study, HFrEF patients were categorized as having persistent inflammation if their plasma hsCRP levels were >2 mg/L.

### Study Details

Eligible NYHA Class II/III patients were enrolled in the Double-blind, Randomized, Sham-procedure-controlled, Parallel-Group Efficacy and Safety Study of Allogeneic Mesenchymal Precursor Cells (Rexlemestrocel-L) in Chronic Heart Failure Due to LV Systolic Dysfunction (Ischemic or Nonischemic) (DREAM HF-1) trial. HFrEF patients were administered: (1) MPCs cultured in 10% fetal serum (n=37), (2) MPCs cultured in the presence of newborn calf serum (5% FCS/5% NBCS; n=153) or, (3) a sham control (i.e., no MPCs; n=241). As evidenced by the serum analysis described in Example 4, cells cultured in media supplemented with newborn serum were effectively cultured in media comprising increased levels of pro-inflammatory cytokines. Cells were administered in a single transendocardial injection. LV systolic function in HFrEF was measured by echocardiogram (ECHO) parameters including left ventricular ejection fraction (LVEF; %), left ventricular end-systolic volume (LVESV; mL), and left ventricular end-diastolic volume (LVEDV; mL) at baseline and 12 months post treatment. Plasma CRP levels were measured to determine baseline levels of inflammation.

### Results

MPCs cultured in the presence of newborn calf serum (5% FCS/5oNBCS) were found to improve left ventricular (LV) systolic function in HFrEF patients at 12 months. In particular, newborn serum-cultured MPCs significantly increased LVEF and decreased LVESV compared to sham controls (p=0.0398 and 0.0426, respectively) (FIG. 2).

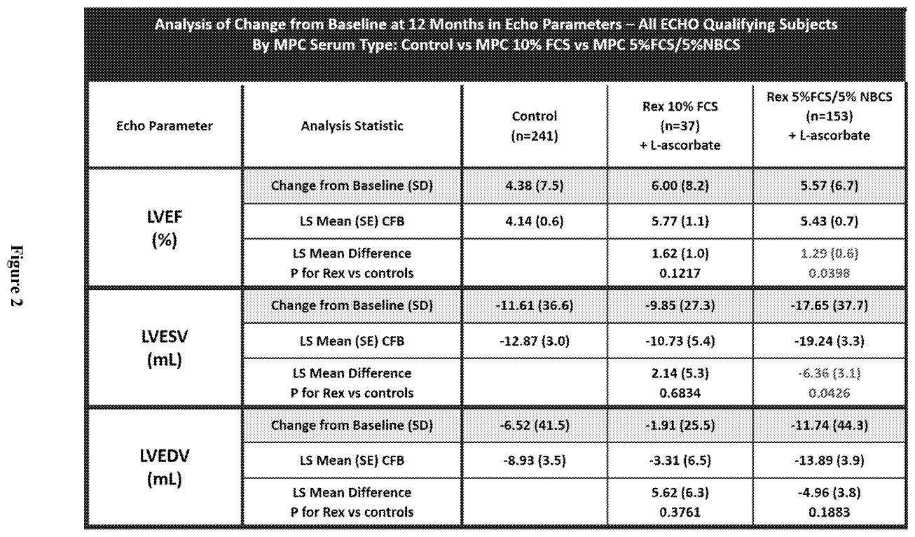

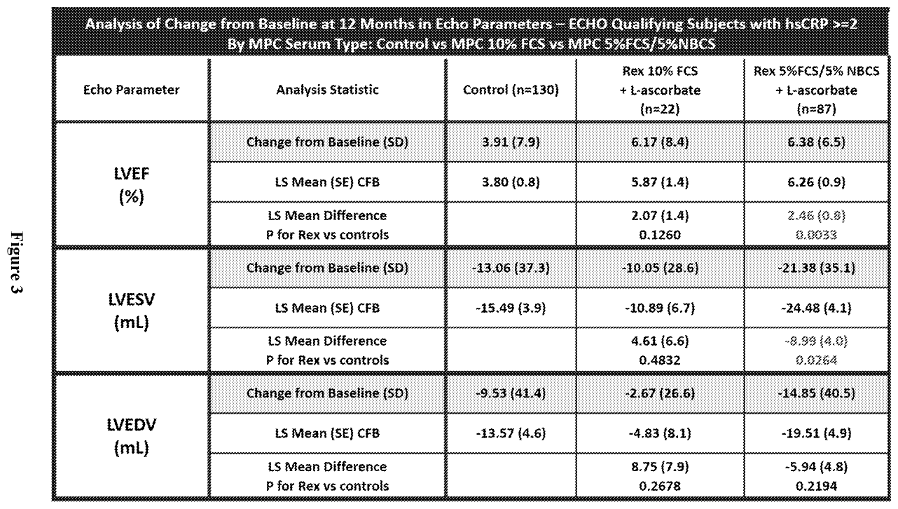

HFrEF patients were then characterised according to plasma hsCRP levels of either <2 mg/L (normal baseline systemic inflammation) or >2 mg/L (elevated baseline systemic inflammation). Importantly, when HFrEF patients were differentiated according to baseline systemic inflammation status (CRP>2), the effect of treatment with MPCs cultured in the presence of newborn calf serum (5% FCS/5% NBCS) LV systolic functional recovery induced was more pronounced. In contrast, MPCs cultured in 10% FBS did not induce a significant effect (FIG. 3). Newborn serum-cultured MPCs significantly increased LVEF % by an average (LS mean) of 2.46 and decreased LVESV by an average of 8.99 mL compared to sham controls (p=0.0033 and 0.0264, respectively). In contrast, MPCs cultured in 10% fetal serum or 5%/FCS/5% NBCS showed improvements to LV systolic function in HFrEF patients without elevated baseline inflammation (HFrEF patients with CRP<2) (FIG. 4).

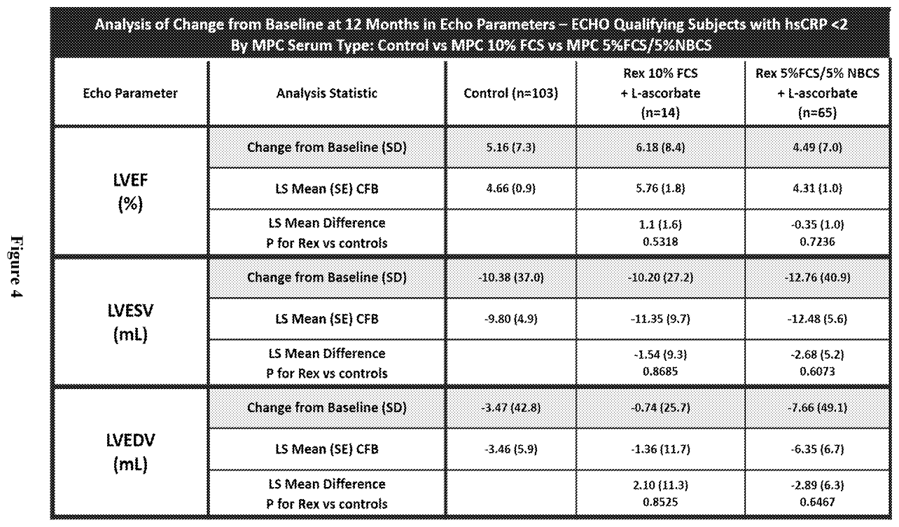

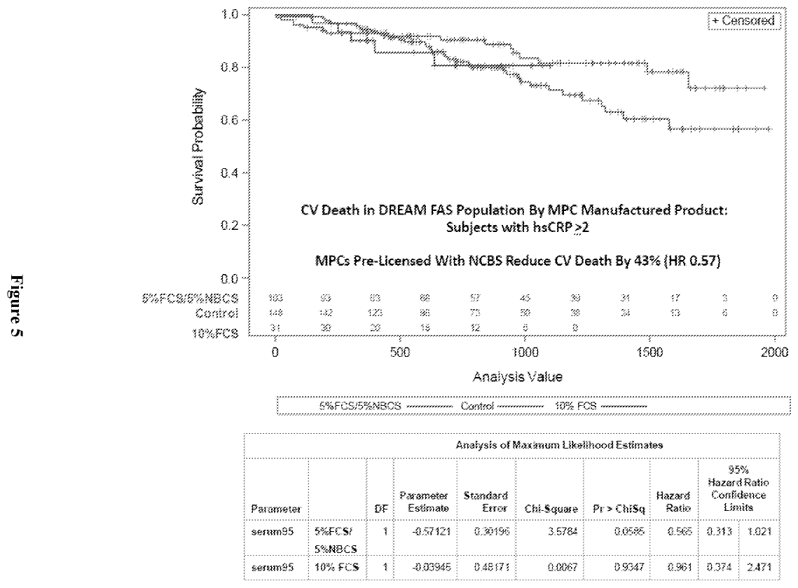

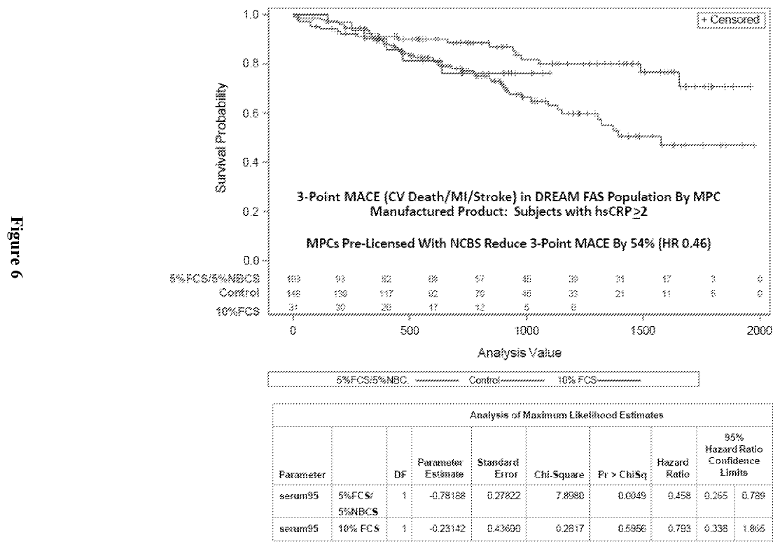

MPCs cultured in newborn serum were also found to reduce other cardiac outcomes in HFrEF patients with CRP>2, including reducing the risk of cardiovascular death by 43% (FIG. 5) and incidence of 3-point MACE (CV Death/MI/Stroke) by 54% (FIG. 6). These data show that MPCs cultured in newborn serum provide improved therapeutic efficacy in the context of persistent inflammation

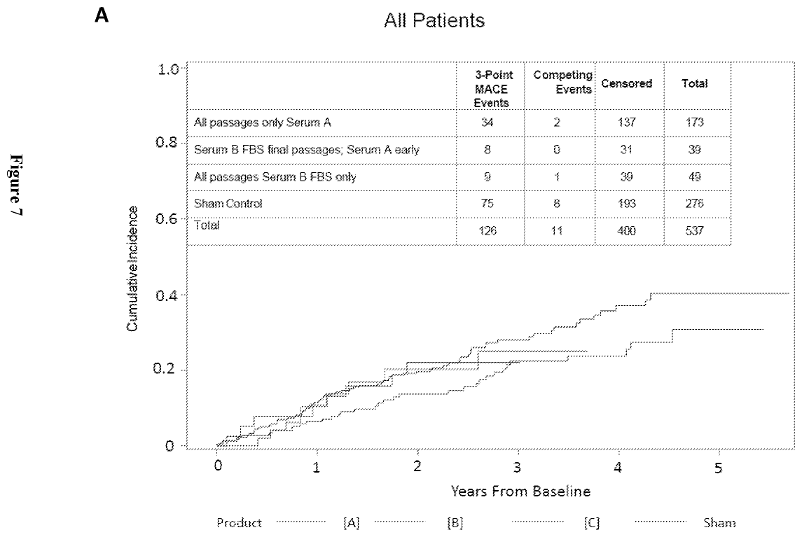

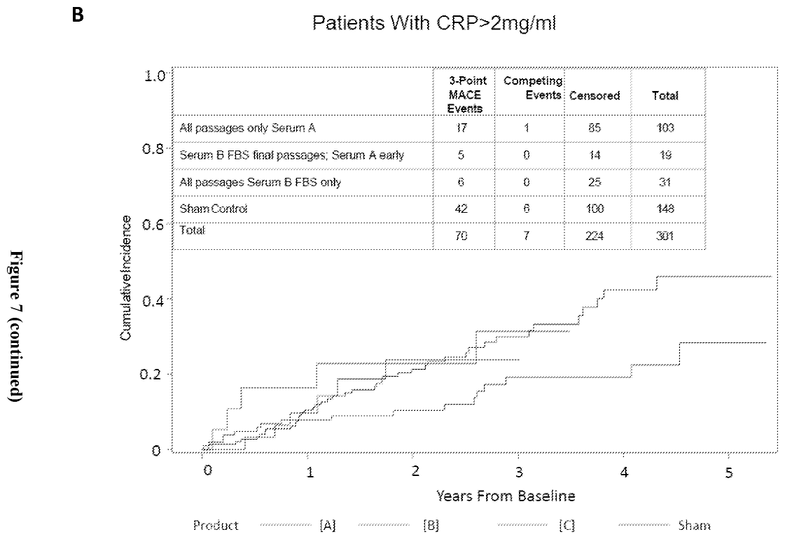

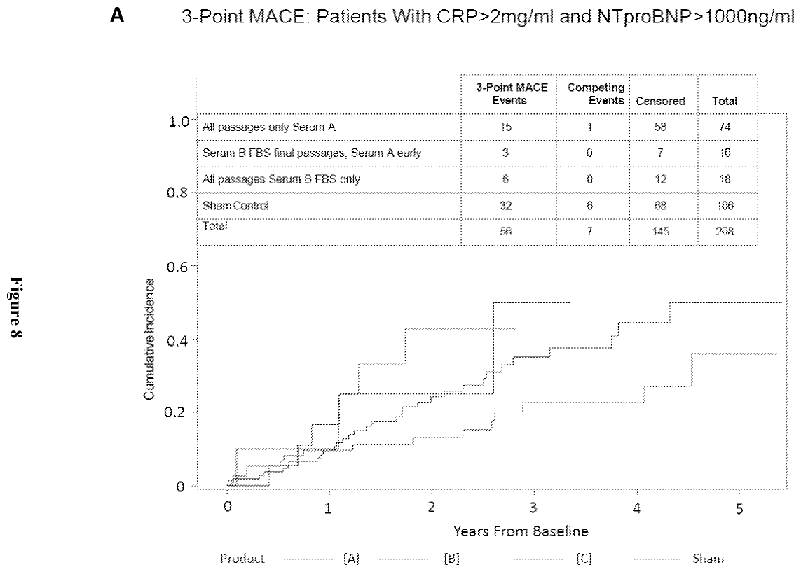

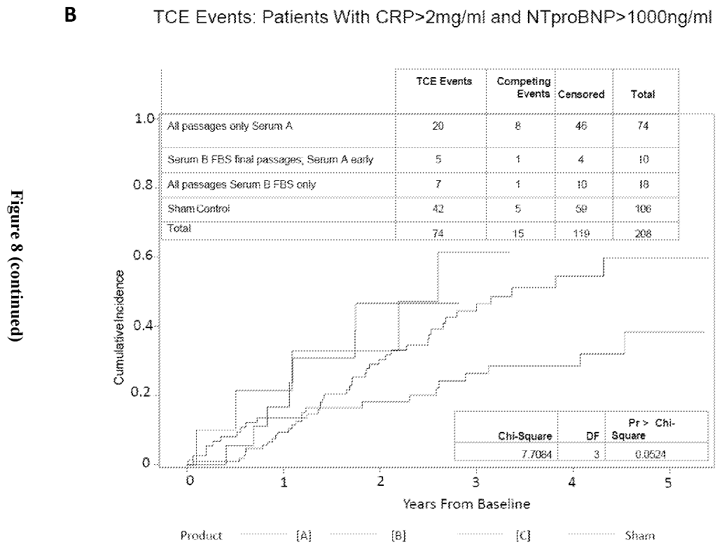

Further analysis of clinical response surprisingly revealed the importance of culture expanding MLPSCs in media supplemented with newborn serum and/or pro-inflammatory cytokines during final passage(s) before administration. MLPSCs cultured in media supplemented with newborn serum and/or pro-inflammatory cytokines during final passages reduced 3-Point MACE (MI, Stroke or CV Death) in patients, irrespective of whether the MLPSCs were culture expanded in FBS during earlier passages. Surprisingly, this reduction in 3-point MACE was observed in all patients, regardless of inflammation status (FIG. 10). Patient sub group analysis revealed that the reduction in 3-point MACE was observed in patients with persistent inflammation (CRP>2 mg/ml; FIG. 7) and, patients in the study at highest risk of Terminal Cardiac Events (TCE; defined as CV Death, Heart Transplant or Left Ventricular Assists Device Implantation). The patients at highest risk of TCE are defined by both CRP>2 mg/ml and NT-proBNP>1000 ng/ml; FIG. 8)).

In contrast, increased adverse events (3-Point MACE) were observed in all patients administered MLPSCs that were culture expanded in media that was not supplemented with newborn serum and/or pro-inflammatory cytokines during final passages before administration. The increase in 3-point MACE was observed 5 regardless of whether the MLPSCs were cultured in media supplemented with newborn serum and/or pro-inflammatory cytokines during earlier passages.

### SUMMARY

MPCs expanded in media supplemented with newborn calf serum (NBCS) and/or pro-inflammatory cytokines:
improved left ventricular systolic dysfunction in HFrEF patients with inflammation, as measured by LS mean change in LVEF and LVESV at 12 months;
reduced cardiovascular death by 43% in high-risk NYHA Class II/III patients with HFrEF and inflammation; and,
reduced long-term 3 Point MACE by 54% in high-risk NYHA Class II/III patients with HFrEF and inflammation.

Taken together with the results in Example 4, these human trial data indicate that supplementing cell culture media with newborn serum and/or pro-inflammatory cytokines provides a cell population with different functional characteristics, at least in terms of capacity to direct therapeutic efficacy in an inflammatory environment.

Without wishing to be bound by any particular theory, these data suggest that supplementing culture media supplemented with proinflammatory cytokines and/or NCBS pre-licenses MPCs to respond more effectively to inflammatory environments, and facilitate partial reprogramming of cells in vivo consistent with the ability of MPCs to inhibit NK cell activation and NK-mediated inhibition of partial reprogramming.

The findings of the present inventors underpin broad application of pre-licensed MPCs (e.g. MPCs cultured media supplemented with proinflammatory cytokines and/or NCBS) for enhancing partial reprogramming and for treatment of any disease or disorder characterised by elevated inflammation, in particular diseases characterized by persistent inflammation such as heart failure.

### Example 8: MLPSCs Cultured in Media Supplemented with Newborn Fetal Serum are Effective in Treating GvHD

10 GvHD patients were administered (intravenous) MPCs culture expanded with NBCS containing pro-inflammatory cytokines (Examples 4 and 6) once per week at a dose of 2×10⁶ MPCs per kg. Patient response is summarised in Table 3. 80% of GvHD patients administered MPCs cultured in NBCS responded to treatment. 1 patient achieved complete response, 7 patients achieved partial response and 2 patients died.

TABLE 3
GvHD response to MPCs culture expanded with NBCS.

| | Non-fetal serum |
|---|---|
| Complete Response (CR) | 1 |
| Partial Response (PR) | 7 |
| Died | 2 |
| No Response (NR) | 0 |
| Total | 10 |

Taken together with the results of at least Example 5, these data further evidence the anti-inflammatory properties of MLPSCs in a T-cell mediated disorder. Accordingly, there is good evidence underpinning the use of MLPSCs, in particular those MLPSCs that have been culture expanded in media supplemented with pro-inflammatory cytokines and/or newborn serum, in partial reprogramming.

1. A method for enhancing partial reprogramming of target cells in a subject in need thereof, the method comprising administering a plurality of mesenchymal lineage progenitor or stem cells (MLPSCs), exosomes derived therefrom, or conditioned media derived from culture of a plurality of MLPSCs to a subject that expresses or will express, following the administration, one or more reprogramming factors in a population of target cells, whereby a plurality of the target cells in the subject become partially reprogrammed.

2. The method according to claim 1, wherein the population of target cells comprises one or more exogenous nucleic acids encoding the one or more reprogramming factors.

3. The method according to claim 1, wherein the population of target cells comprises one or more exogenous nucleic acids encoding one or more targeted transactivators for inducing expression of endogenous genes encoding the one or more reprogramming factors.

4. The method according to any one of claims 1 to 3, wherein the MLPSCs are culture expanded from a population of STRO-1⁺ mesenchymal progenitor cells (MPCs) or mesenchymal stem cells (MSCs).

5. The method according to any of claims 1 to 4, wherein the MLPSCs are pre-licensed MLPSCs.

6. The method according to claim 5, wherein the MLPSCs are MLPSCs that were culture expanded in media containing:
IFN-gamma and/or TNF-alpha; and
one or more pro-inflammatory cytokines selected from the group consisting of IL-6, IL-8, IL-17A, MCP-1-alpha, MIP-1-beta, and IP-10.

7. The method according to claim 6, wherein the media contains serum that comprises IFN-gamma and/or TNF-alpha; and the one or more pro-inflammatory cytokines.

8. The method according to claim 7, wherein the culture medium comprises newborn mammalian serum.

9. The method according to claim 8, wherein the newborn mammalian serum is newborn calf serum.

10. The method according to any one of claims 1 to 9, wherein the MLPSCs are MLPSCs genetically modified to overexpress indoleamine 2,3-dioxygenase (IDO) and/or cyclooxygenase-2 (COX-2).

11. The method according to any one of claims 1 to 10, comprising administering the plurality of MLPSCs.

12. The method according to any one of claims 1 to 10, comprising administering the exosomes derived from a plurality of MLPSCs.

13. The method according to any one of claims 1 to 10, comprising administering the conditioned media derived from culture a plurality of MLPSCs.

14. The method according to any one of claims 1 to 13, wherein the subject is suffering from or at risk of a health condition selected from the group consisting of: cardiovascular diseases, vascular endothelial conditions, low back pain, inflammatory diseases, osteoarthritis, metabolic diseases, kidney diseases, liver diseases, and neurological disorders.

15. The method according to any one of claims 1 to 14, wherein the target cells are selected from the group consisting of: cardiomyocytes, nucleus pulposus cells, renal cells, liver cells, endothelial progenitors, chondrogenic progenitors, chondrocytes, neurons, neural progenitors, and glial cells.

16. The method according to any one of claims 1 to 15, wherein the one or more reprogramming factors are selected from the group consisting of: OCT4, SOX2, KLF4, c-MYC, LIN28, and NANOG.

17. The method according to any one of claims 1 to 16, wherein the one or more reprogramming factors comprise OCT4, SOX2, and KLF4.

18. The method according to claim 17, wherein the one or more reprogramming factors comprise OCT4, SOX2, KLF4, and c-MYC.

19. The method according to claim 17, wherein the one or more reprogramming factors comprise OCT4, SOX2, and KLF4, but not c-MYC.

20. The method according to claim 16, wherein the one or more reprogramming factors: (i) do not comprise LIN 28; or (ii) do not comprise NANOG.

21. The method according to claim 16, wherein the one or more reprogramming factors comprise OCT4, SOX2, KLF4, c-MYC, LIN28, and NANOG.

22. The method according to claim 21, wherein the one or more reprogramming factors:
(i) consist of OCT4, SOX2, KLF4, c-MYC, LIN28, and NANOG;
(ii) OCT4, SOX2, KLF4, and c-MYC; or
(iii) OCT4, SOX2, and KLF4.

23. The method according to any one of claims 2 to 22, wherein the one or exogenous nucleic acids comprise one or more synthetic mRNAs encoding the one or more reprogramming factors or encoding the one or more targeted transactivators.

24. The method according to claim 23, wherein the one or more synthetic mRNAs comprise one or more nucleoside-modified mRNAs.

25. The method according to any one of claims 2 to 22, wherein the one or more exogenous nucleic acids comprise one or more non-integrated expression cassettes for expression of the one or more encoded reprogramming factors or targeted transactivators in mammalian cells.

26. The method according to claim 25, wherein the one or more non-integrated expression cassettes are provided as one or more: plasmid expression vectors, minicircle expression vectors, linear amplicons, circular DNA amplicons, recombinant DNA viral genomes or amplicons, or recombinant RNA viral genome, amplicons, or any combinations thereof.

27. The method according to claim 25 or claim 26, wherein expression of at least one of the one or more encoded reprogramming factors or targeted transactivators is under the control of a target cell-selective promoter.

28. The method according to claim 27, wherein the target cell-selective promoter is selected from the group consisting of: cardiomyocyte-selective promoters, nucleus pulposus cell-selective promoters, renal cell-selective promoters, liver cell-selective promoters, endothelial progenitor-selective promoters, chondrogenic progenitor-selective promoters, glial cell-selective promoters, neural progenitor-selective promoters, and neuron-selective promoters.

29. The method according to any one of claims 2 to 28, wherein at least one of the exogenous nucleic acids is polycistronic.

30. The method according to any one of claims 1 to 29, wherein expression of the one or more reprogramming factors is inducible and reversible.

31. The method according to any one of claims 1 to 30, wherein the MLPSCs are modified MLPSCs comprising at least one of: an exogenous anti-inflammatory: miRNA, antagomiR, siRNA, RNAi, antisense oligonucleotide, or an antisense RNA.

32. The method according to any one of claims 1 to 31, wherein the MLPSCs are modified MLPSCs comprising at least one of an exogenous siRNA, RNAi, antisense oligonucleotide, or antisense RNA directed against at least one of: p16^{INK4a}, p21^{WAF1/CIP1}, the mTOR signalling pathway, or the c-Jun N-terminal kinase (JNK) signalling pathway.

33. The method according to any one of claims 1 to 32, wherein the one or more reprogramming factors are expressed consecutively for at least about two, days but not longer than about 15 days.

34. The method according to claim 33, wherein the one or more reprogramming factors are expressed for a cumulative period of at least two days, but not longer than about 15 days.

35. The method according to any one of claims 1 to 34, wherein the plurality of MLPSCs are administered prior to initial expression of the one or more reprogramming factors.

36. The method according to any one of claims 1 to 34, wherein the plurality of MLPSCs are administered after expression of the one or more reprogramming factors has already begun.

37. The method according to any one of claims 2 to 36, further comprising delivering the one or more exogenous nucleic acids to the population of target cells.

38. The method according to any one of claims 1 to 37, further comprising administering prostaglandin E₂ to the subject.

39. The method according to any one of claims 1 to 38, further comprising administering a blocking antibody to the Natural Killer cell receptor NKG2D.

40. A method for enhancing partial reprogramming of target cells in a post-natal subject in need thereof, the method comprising administering a plurality of mesenchymal lineage progenitor or stem cells (MLPSCs), exosomes derived therefrom, or conditioned media derived from culture of the plurality of a plurality MLPSCs to a subject that expresses, concomitantly with the administration or after the administration, exogenous reprogramming factors comprising OCT4, SOX2, KLF4, and c-MYC in a population of target cells, wherein one or more exogenous nucleic acids for expression of the exogenous reprogramming factors have been delivered to the target cell population (i) as one or more synthetic mRNAs; (ii) by one or more non-integrating recombinant viruses; or (iii) as a combination of (i) and (ii); whereby a plurality of the target cells in the post-natal subject become partially reprogrammed.

41. The method according to claim 40, wherein the one or more exogenous nucleic acids are delivered to the target cell population as one or more synthetic mRNAs.

42. The method according to claim 40, wherein the one or more exogenous nucleic acids are delivered to the target cell population by one or more non-integrating recombinant viruses.

43. The method according to claim 42, wherein the non-integrating recombinant virus is selected from the group consisting of adenovirus, adeno-associated virus, non-integrating lentivirus, human cytomegalovirus (CMV), herpes simplex virus (HSV), and Sendai virus.

44. The method according to any one of claims 40 to 43, wherein the MLPSCs are administered concomitantly with expression of the exogenous reprogramming factors.

45. The method according to any one of claims 40 to 43, wherein the MLPSCs are administered prior to expression of the exogenous reprogramming factors.

46. The method according to any one of claims 40 to 45, further comprising delivering to the target cell population the one or more exogenous nucleic acids for expression of the exogenous reprogramming factors.

47. The method according to any of claims 40 to 46, wherein the MLPSCs are pre-licensed MLPSCs.

48. The method according to claim 47, wherein the MLPSCs are MLPSCs that were culture expanded in media containing:
IFN-gamma and/or TNF-alpha; and,
one or more pro-inflammatory cytokines selected from the group consisting of IL-6, IL-8, IL-17A, MCP-1-alpha, MIP-1-beta, and IP-10.

49. The method according to claim 48, wherein the media contains serum that comprises IFN-gamma and/or TNF-alpha; and the one or more pro-inflammatory cytokines.

50. The method according to claim 49, wherein the serum is newborn mammalian serum.

51. The method according to claim 50, wherein the newborn mammalian serum is newborn calf serum.

52. The method according to any one of claims 40 to 51, wherein the MLPSCs are administered by a local route of administration.

53. A population of MLPSCs when used in the method according to any one of claims 1 to 52.

54. A composition for enhancing partial reprogramming of target cells, comprising a plurality of modified mesenchymal lineage progenitor or stem cells (MLPSCs) comprising one or more exogenous nucleic acids encoding one or more: (a) reprogramming factors; or (b) targeted transactivators that induce expression of one or more endogenous reprogramming factors, wherein:
(i) the modified MLPSCs, when in the presence of one or more target cells, deliver the one or more exogenous nucleic acids to the one more target cells;
(ii) the one or more encoded reprogramming factors are expressed in the one or more target cells at a level sufficient to partially reprogram the one or more target cells; and
(iii) the one or more encoded reprogramming factors or targeted transactivators are substantially inoperable to expression in the modified MLPSCs prior to the delivery in (i).

55. A composition for enhanced partial reprogramming of target cells, comprising:
(i) a culture-expanded population of human mesenchymal stem cells (hMSCs);
(ii) a conditionally-replicating helper virus; and
(iii) one or more helper-dependent viruses that: (a) comprise one or more expression cassettes for one or more reprogramming factors or targeted transactivators that induce expression of one or more endogenous reprogramming factors; and (b) substantially free of viral coding sequences.

56. The composition according to claim 55, wherein the hMSCs are transduced with (ii), (iii), or both (ii) and (iii).

57. The composition according to claim 55 or claim 56, wherein the one or more expression cassettes are inducible and reversible expression cassettes.

58. The composition according to claim 57, wherein the conditionally replicating helper virus or the one or more helper-dependent viruses encode a regulatable transactivator that controls expression from the one or more inducible and reversible expression cassettes.

59. The composition according to any one of claims 55 to 58, wherein the conditionally-replicating helper virus and the one or more helper-dependent viruses are adenoviruses, herpes simplex viruses (HSVs), non-integrating lentiviruses, or adeno-associated viruses (AAV).

60. The composition according to claim 59, wherein the conditionally-replicating helper virus and the one or more helper-dependent viruses are adenoviruses.

61. The composition according to any one of claims 55 to 60, wherein the culture-expanded hMSCs are pre-licensed hMSCs.

62. A pharmaceutical composition comprising the composition according to any one of claims 54 to 61 and a pharmaceutically acceptable excipient.

63. The pharmaceutical composition according to claim 62, further comprising one or more of prostaglandin E₂ or a blocking antibody to the Natural Killer cell receptor NKG2D.

64. A method for enhanced partial reprogramming of target cells, the method comprising administering to a subject in need thereof the composition according to any one of claims 56 to 63.

65. The method according to claim 64, wherein the subject is suffering from or at risk of a health condition selected from the group consisting of: cardiovascular diseases, vascular endothelial conditions, low back pain, inflammatory diseases, osteoarthritis, metabolic diseases, kidney diseases, liver diseases, and neurological disorders.

66. The method according to claim 64 or claim 65, wherein the transduced hMSCs are administered by a local route of administration.
